(19) United States
(12) Patent Application Publication
(10) Pub. No.: US 2024/0011925 A1
(43) Pub. Date: Jan. 11, 2024

(54) NONINVASIVE WATER CONTENT SENSOR

(71) Applicant: Southern Methodist University, Dallas, TX (US)

(72) Inventors: Jungchih Chiao, Grand Prairie, TX (US); Sen Bing, Dallas, TX (US)

(21) Appl. No.: 18/342,881

(22) Filed: Jun. 28, 2023

Related U.S. Application Data
(60) Provisional application No. 63/358,423, filed on Jul. 5, 2022.

(51) Int. Cl. G01N 22/04; G01N 33/18; A61B 5/00

(52) U.S. Cl. CPC G01N 22/04; G01N 33/18; A61B 5/6847; H01Q 7/005

(57) ABSTRACT

A system for noninvasive monitoring of water content comprising a radio-frequency (RF) planar resonant loop sensor comprising a planar loop antenna; and an element disposed within and co-planar with a loop formed by the planar loop antenna; and a vector network analyzer configured to be connected with the RF planar resonant loop sensor.

### CROSS-REFERENCE TO RELATED APPLICATIONS

This application claims priority based on U.S. Provisional Application No. 63/358,423, filed Jul. 5, 2022. The contents of which is incorporated by reference in its entirety.

### STATEMENT OF FEDERALLY-FUNDED RESEARCH

This invention was made with government support under ENG-CMMI-1929953 awarded by the National Science Foundation. The government has certain rights in the invention.

### TECHNICAL FIELD OF THE INVENTION

The present invention relates in general to resonant coupler systems and methods. In particular, the present invention relates to the resonant coupler systems and methods for implant devices and associated devices.

### BACKGROUND OF THE INVENTION

Without limiting the scope of the invention, its background is described in connection with resonant coupler systems and methods.

Modern electronic implants have advanced functions and reduced sizes significantly by integration of low-power electronics. Targeting better management of chronic diseases, sensing, recording, and electrical stimulation have been considered to be incorporated into a single device, which will require signal and data communication, remote control, and battery charging capabilities. Furthermore, battery size and capacity can be greatly reduced by efficient wireless charging, or the battery can be completely eliminated with wireless powering. With supercapacitors instead of a battery [1] [2], electric circuits and electrodes can be made on a flexible substrate and packaged with lamination. This opens a new class of implants that can be sufficiently thin and flexible to be implemented subcutaneously or interstitially for electrical and electrochemical sensing or stimulation [3]-[7].

Conventional wireless power transfer methods for implants use coils for inductive coupling [8]-[13]. It has been implemented for the charging function in several FDA-approved neurostimulators, including some in clinical studies. Commercially available neurostimulators have been compared on their efficacies and costs in practical uses [14]-[21]. Coil antennas use magnetic field coupling to generate electric currents, as compared to antennas based on electric field coupling, which can provide higher transfer powers. Electric field coupling antennas are constrained in designs by their dimensions in scales of the wavelengths at their operating frequencies. Magnetic coupling with a sufficiently high mutual inductance, typically achieved by large self-inductance with high turn numbers of coils, can achieve good power coupling without the limitation related to wavelength. In pacemakers or neuro-stimulators, the cross-section of coils is kept limited in order to keep the implants compact so that the incision for implantation procedures can be small to prevent unnecessary patient pain. Typical stimulators have volumes of 14-40 cm³ [22]. The smallest FDA-approved stimulators have a conventional shape with a 47.2×57.1 mm cross-section, so the coil cross-section is then limited [14] [23]. The coil antenna can be larger for the external handheld device, acting as a reader and power transmitter. However, the dimensions are still limited by operation convenience as the patient has to hold it by hand against the chest or waist to charge the implant for tens of minutes or even a few hours.

Typically, wireless power charging at the resonant frequency provides better efficiency. A wound-wire solenoid with a known self-inductance is matched with a tuning capacitor to achieve resonance at the desired operating frequency. Often, the inductance is determined empirically. Communication can be conducted through the same coils because the data rate for vital sign information and control command is usually low. Such solenoid coils intrinsically are bulky due to winding wires and its 3-D architecture. The operation is limited to lower frequencies along with low quality factors, not to mention it does not allow planar or monolithic configurations of implants.

As the power transfer occurs in the near-field range, the coupling coefficient and mutual inductance vary the loading impedance to the implant coil, creating an impedance mismatch for the circuit of the entire system. This affects the transmission and reflection coefficients in the implant and transmitter sides. Although dynamic tuning can adjust the reader/transmitter for better impedance matching, it is not preferred to have automatic or manual tuning in the implant in order to avoid additional circuit complexity or size increases. This is particularly critical for planar subcutaneous or interstitial implants.

Furthermore, with the trend that implant sizes are getting smaller and future implants are moving toward planar configurations, a quick and convenient way to identify the device location inside the body is needed, especially for subcutaneous implants. The conventional way of X-ray computed tomography [24] [25] takes time, adds more costs, and exposes patients to additional risks since it is by ionizing radiation.

For subcutaneous and interstitial implants, the thickness of the device package should be as thin as possible. For example, subcutaneous electrodes are implemented for electrical stimulation of peripheral nerves to inhibit chronic pain [5]. It demonstrated substantially reduced procedural risk and improved quality of life by reducing pain without analgesia; however, the wired connection presented practicality issues for long-term uses. A permanent subcutaneous implant with wireless control and power will resolve the usability issue to optimize pain management benefits. Another example is a foldable gastrostimulator fabricated on a polyimide substrate that can be folded into a cylinder shape and inserted into a tube delivered by an endoscope into the stomach via mouth and esophagus [3]. The device is then unfolded back to its planar shape and inserted into the stomach's submucosal layer as a secure attachment method. The gastrostimulator delivers electrical pulses into the mucosal and submucosal layers of the stomach to modulate its motility. These devices are based on flexible biocompatible polyimide substrates, and planar spiral antennas are utilized for inductive coupling [7]. In such cases, the turn numbers are limited due to the substrate size. As an effort to increase self-inductance, reduction of metal line width in order to increase total metal line length inevitably increases AC resistance of the coil and reduces the operating frequency. Furthermore, the effective permittivity experienced by the antenna changes with the implant depth. Impedance matching becomes challenging because the circuits need to provide a wide range of tuning capacitances to minimize reflection losses at different implant depths under the skin.

Microwave sensors have been utilized for water sensing owing to the distinctive dielectric properties of water at high frequencies for remote sensing applications [50]. Three main types of microwave measurement methods are used: resonance, transmission, and reflection. Resonance cavities can accurately measure the dielectric properties of a medium, obtaining frequency characteristics owing to the interference in the medium. However, measurement is limited for media with high dielectric losses [51]. The physical constructures of resonators have constraints to be used on the human body. The transmission measurements require both transmitter and receiver antennas, by which electric fields pass through and interact with the media. Garrett et al. developed a transmission sensor to monitor human hydration levels by evaluating the effective permittivity in the forearm with microwave signals passing the tissues [52]. Such a system can be bulky, and the high electric fields may have safety concern. The reflection type has simpler physical structures and can be potentially designed as a wearable for long-term continuous monitoring. However, near-field characteristics, sizes, and wave scattering for planar antennas face design challenges. The radiation powers also affect the interference effects at different depths inside tissues.

Despite these advances, a need remains for novel, affordable, and effective, generally contactless water monitoring systems and methods.

### SUMMARY OF THE INVENTION

As embodied and broadly described herein, an aspect of the present disclosure relates to a system for noninvasive monitoring of water content comprising: a radio-frequency (RF) planar resonant loop sensor comprising: a planar loop antenna; and an element disposed within and co-planar with a loop formed by the planar loop antenna; and a detector configured to be connected with the RF planar resonant loop sensor to detect a near-field resonance. In one aspect, the water is disposed within organic material comprising human tissue, non-human animal tissue, or plant tissue. In another aspect, the water is disposed within an inorganic material. In another aspect, the system is configured to be disposed on or about a surface. In another aspect, the detector comprises a vector network analyzer, a scalar network analyzer, a spectrum analyzer, a phase-lock loop, or a frequency lock circuit. In another aspect, the system is configured to measure an |s₁₁| reflection coefficient. In another aspect, the system is configured to monitor water content over time by measuring a resonance twice or more in a selected time period. In another aspect, the system is configured to monitor water content over time by measuring a resonance continuously during a selected time period.

As embodied and broadly described herein, an aspect of the present disclosure relates to a kit comprising: a radio-frequency (RF) planar resonant loop sensor comprising: a planar loop antenna; and an element disposed within and co-planar with a loop formed by the antenna; and a detector configured to be connected with the RF planar resonant loop sensor to detect a near-field resonance; and a device to secure the RF planar resonant loop sensor to a surface. In one aspect, the water is disposed within an organic material comprising human tissue, non-human animal tissue, or plant tissue. In another aspect, the water of is disposed within an inorganic material. In another aspect, the system is configured to be disposed on or about a surface. In another aspect, detector comprises a vector network analyzer, a scalar network analyzer, a spectrum analyzer, a phase-lock loop, or a frequency lock circuit. In another aspect, the system is configured to measure an |s₁₁| reflection coefficient. In another aspect, the system is configured to monitor water content over time by measuring a resonance twice or more in a selected time period. In another aspect, the system is configured to monitor water content over time by measuring a resonance continuously during a selected time period.

As embodied and broadly described herein, an aspect of the present disclosure relates to a method of measuring water content in a material comprising: providing the material; providing a system for noninvasive monitor or water content comprising: a radio-frequency (RF) planar resonant loop sensor system comprising: a planar loop antenna; and an element disposed within and co-planar with a loop formed by the antenna; and a detector configured to be connected with the RF planar resonant loop sensor to detect a near-field resonance; disposing the loop on a surface of the material; and measuring a near-field resonance with the system. In one aspect, the material comprises an organic material comprising human tissue, non-human animal tissue, or plant tissue. In another aspect, the material comprises an inorganic material. In another aspect, the step of measuring the near-field resonance with the system comprises measuring an |s₁₁| reflection coefficient. In another aspect, the method further comprises measuring a near-field resonance twice or more in a selected time period. In another aspect, the method further comprises measuring a near-field resonance continuously during a selected time period.

### BRIEF DESCRIPTION OF THE DRAWINGS

For a more complete understanding of the features and advantages of the present invention, reference is now made to the detailed description of the invention along with the accompanying figures, in which:

FIG. 1A shows a resonate coupler device architecture.

FIG. 1B shows dimensions of a resonate coupler device.

FIG. 2A shows a photograph of tuned resonate couplers with center pads.

FIG. 2B shows prior art antennas without pads of the Prior Art.

FIG. 3A shows a simulation configuration.

FIG. 3B shows a measurement configuration.

FIG. 4 shows simulation and measurement results of the resonate coupler for the ISM frequency band 902-928 MHz with and without the center pad.

FIG. 5 shows simulation and measurement results for the ISM frequency band 2.4-2.5 GHz with and without the center pad.

FIG. 6 shows quality factors of the tuned loop at the two ISM bands for the implant depth of 6 mm.

FIGS. 7A and 7B show simulations of the reflection coefficient for the 903-MHz (FIG. 7A) and 2.45-GHz (FIG. 7B) designs in the depths of 3, 6, 9 and 12 mm, where the simulations are conducted with human skin properties documented in [29].

FIGS. 8A and 8B show measurements of the 903-MHz design (FIG. 8A) and the 2.45-GHz design (FIG. 8B) at depths of 3, 6, 9 and 12 mm in the ground pork phantom.

FIGS. 9A and 9B show frequency shift percentage (left y-axis) and su magnitude (right y-axis) in the depth of 0-12 mm for the 903-MHz (FIG. 9A) and 2.45-GHz (FIG. 9B) designs, where measurements in discrete depths of 3, 6, 9, 12 mm are compared with simulation results obtained at 1-mm steps.

FIG. 10 shows simulations showing frequency matching after a relative permittivity decrease of 25 and a conductivity decrease of 0.35 S/m.

FIG. 11 shows a comparison of simulation utilizing the documented frequency-dependent human skin electrical properties, measurement on top of a person's forearm skin, and measurement on the top surface of the ground pork cube.

FIG. 12A shows the sensor configuration. FIG. 12B shows a photograph of the assembled sensor. FIG. 12C shows a setup of human hydration experiment with the sensor placed on the forearm and connected to the vector network analyzer.

FIG. 13 shows simulation results of the sensor with (tuned) and without the center pad (untuned).

FIG. 14A shows seven human hydration experiments. FIG. 14B shows resonant frequency shifts in time during the human hydration experiments with five dehydrated participants.

FIG. 15A shows the sensor design and FIG. 15B shows a photograph of the sensor connected with an SMA connector.

FIG. 16 shows simulation (solid line) and measurement (dashed line) results of the sensor.

FIG. 17 shows results of the continuous hydration monitoring. The blue curve indicates the resonant frequency shift.

FIG. 18 shows a setup of continuous and long-term water content monitoring of produce.

FIG. 19 shows continuous and long-term water content monitoring experiments for orange and ground pork patty.

FIG. 20 shows how electromagnetic field penetrate tissues.

FIGS. 21A and 21B show a loop resonator configuration and a loop resonator with the tuning center pad.

FIG. 22 shows simulations of the reflection coefficients conducted with documented human skin properties [63] with (tuned) and without the center pad (untuned), compared to measurements.

FIG. 23 shows a comparison of electric fields for the loop resonator with or without the tuning pad.

FIGS. 24A and 24B show an architecture of the connection-improved sensor and a photograph of the sensor connected with an SMA connector.

FIG. 25 shows comparisons of simulation results utilizing human dry and wet skin properties documented in the IFAC library [63] and measurements on the forearm when participant was dehydrated or hydrated.

FIG. 26 shows a setup of skin dielectric properties measurement using the dielectric constant probe kit.

FIG. 27A shows comparisons of dielectric constants measured with the probe, compared to the IFAC library dataset [63], with the error bars in the measured results obtained from four measurements, and FIG. 27B simulations of the reflection coefficients in the tuned sensor loop by using the IFAC documented human skin and by the measured dielectric constants on the skin with the probe.

FIG. 28A shows measurements of dielectric properties for dehydrated and hydrated human skins, with the error bars obtained from four measurements at the same location on the forearm, and FIG. 28B shows simulated reflection coefficients for the tuned resonator utilizing the measured human skin dielectric properties, compared to measured results by the tuned loop.

FIG. 29 shows a setup of the human hydrating monitoring measurements.

FIG. 30 shows the resonant frequency shift in time during the human hydration for five dehydrated subjects, where the resonant frequency was tracked along with the resonant coefficient at an interval of 20 seconds continuously.

FIG. 31 shows the resonant frequency shift in time during the human hydration experiment, where the data recording began before the dehydrated participant's intake of water (50 ml volume).

FIGS. 32A and 32B show a photo of the ground pork phantom used in the experiment and a setup of the long-term water content monitoring experiments.

FIG. 33 shows the resonant frequency shift as a function of time during the dehydration experiments using ground pork phantoms.

FIG. 34 shows the total frequency shift in percentage as a function of the weight loss in percentage in the phantom after 19 hours of water evaporation.

FIG. 35A shows the start and end resonant frequencies with their respective effective dielectric constants calculated (FIG. 35B), where the relationship with a linear regression R²=0.919 showed how the effective dielectric constant changed with the water losses.

FIG. 36 shows simulations of resonant frequencies for the phantom in a range of dielectric constants varied for 5 to 60 due to water content changes.

FIG. 37 shows simulations of the resonant frequency for models with different curvature radii, where the model permittivities of dehydrated and hydrated states were measured by a dielectric probe on a human body.

FIG. 38 shows the electric field distribution of the tuned loop: (upper panel) the top view in the plane of the tuned loop, where the side view is taken in the cross section (dashed line), and (lower panel) the side view into the tissues.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,”. “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

The present invention uses planar loop antennas for noninvasively monitoring water content in objects, such as tissues, inside containers, and plant tissue. For example, water plays a vital role in the human and animal bodies, such as transporting nutrients and waste across cells as a carrier and maintaining a stable body temperature in different environments [35]. Significant loss of body water causes dehydration and severe health problems. Physiologically, dehydration contributes to a higher risk of musculoskeletal injury due to the impaired anaerobic muscular power [36] and decreased cardiac outputs with reduced blood volumes [37]. Mentally, dehydration exacerbates cognitive performance and mood [38], making it hard to concentrate with feelings of fatigue and nervousness [39-44]. Generally, the urinary system primely balances the regulation of hydration. Excess body water is excreted by the urinary system, and urine volume is reduced when the body gets dehydrated [45]. The thirsty feeling is activated to remind the person to take in fluid [46]. However, such feedback mechanisms may be impaired for specific populations and scenarios, including infants, the elderly, soldiers, and athletes [47-49]. It is critically essential to continuously, noninvasively, and efficiently monitor the hydration level of those who may be at risk. Similarly, it is necessary to monitor, in real time, the levels of water or moisture within containers (such as food or other containers), of fruits, plants, vegetables, eggs, or other consumable items (e.g., salt, sugar, flour) within containers, without opening the container or breaching the skin or surface of the fruits, plants, vegetables, or eggs.

An embodiment of the present invention includes a modified loop antenna structure for near-field power transfer and data communication, given the aforementioned limitations. The antenna is based on a simple planar loop, of which spectral characteristics can be calculated by the analytical forms in [27], with a metal element embedded for tuning purposes. The center element behaves as distributed reactive tuning elements contributing shunt capacitances and mutual inductances to the impedance of the loop and matching the port impedance at the desired resonant frequency. In various embodiments, the element is a loop element or a pad element. References to a pad herein are merely exemplary and encompass both a pad element and a loop element. Further, in various embodiments, the antenna and the element are circular, elliptical, square-shaped, rectangle-shaped, polygon-shaped, or have other shapes.

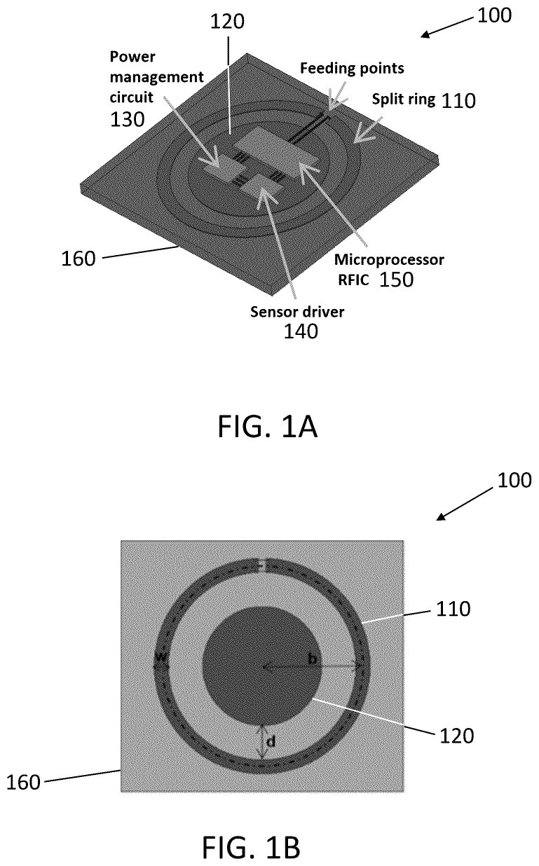

The center element also serves as the space to accommodate circuits making the implant compact, as shown in FIG. 1A. The inventors demonstrates the design strategy for impedance matching with two antennas at 903 MHz of the first resonance and at 2.45 GHz of the second resonance. The impedance matching performance at different implantation depths under the skin is studied. The discrepancies due to the differences of dielectric properties in the documented human skins and phantoms made of ground pork are investigated. The resonant antenna can also serve as a beacon for locating the implant noninvasively by a scanner placed on the skin. The capability and resolution are examined.

In FIG. 1A, an embodiment of the present invention is shown, a planar inductive resonant coupler 100 including a circular planar loop antenna 110 (also referred to herein as a split ring antenna), an element 120 (also referred to herein as a center element), and various exemplary items of circuitry including a power management circuit 130, a sensor driver 140, and a microprocessor radio-frequency integrated circuit (RFIC) 150; and a coupler substrate 160 (also referred to as a substrate 160 herein) on which the antenna 110 and the element 120 may be disposed. The antenna 110, the element 120, or both include metal or some other conductive material in embodiments of the present invention; references herein to a metal antenna or a metal element are exemplary. The pad element shown in FIG. 1A and in any other figure and discussed herein is exemplary of pad and loop elements of various embodiments and is not limiting. Further circular shapes shown in FIG. 1A, and in any other figure and discussed herein, is exemplary of antenna and element shapes of various embodiments and are not limiting.

The resonator consists of a split ring and a center element, shown in FIG. 1B The center element adds distributed capacitances, as electric fields are established across the gap between metal patterns along the loop. The element decreases self-inductance in the loop, as the induced currents on the center element produce opposite-polarization magnetic fields in the gap. The spacing d between the metal loop and the element tunes the distributed reactive elements, and the resultant impedance affects the reflection coefficient s₁₁ at the driving port. An embodiment of the present invention shown in FIG. 1B includes the coupler 100, including an antenna 110 with an element 120, disposed on a coupler substrate 160. Dimensions are shown in FIG. 1B, including w, a width of the split ring; b, a distance between the center axis of the element and the circle halfway between the antenna inner diameter and the outer antenna diameter; and d, the width of the gap between the diameter of the element and the antenna inner diameter. Two designs are conducted for the ISM bands of 902-928 MHz and 2.4-2.5 GHz. For the operating frequency at 903 MHz, b=13.15 mm and w=2a=1.8 mm. The design has b=8.9 mm and 2a=1.16 mm for the carrier frequency of 2.45 GHz. In both cases, Ω is kept at 9, which gives reasonable fabrication tolerance to compare measurements and theory.

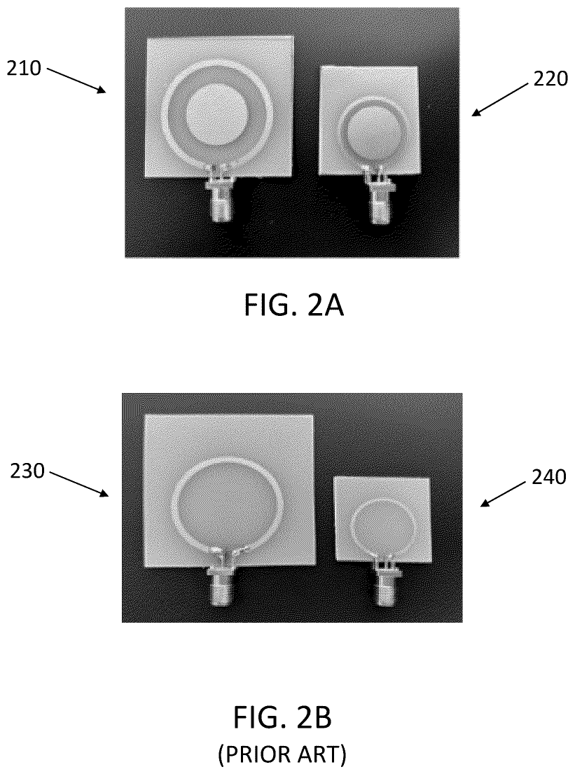

The test articles are shown in photographs in FIG. 2A: the test article 210 for 903 MHz and the test article 220 for 2.45 GHz. FIG. 2B shows prior art antennas without elements.

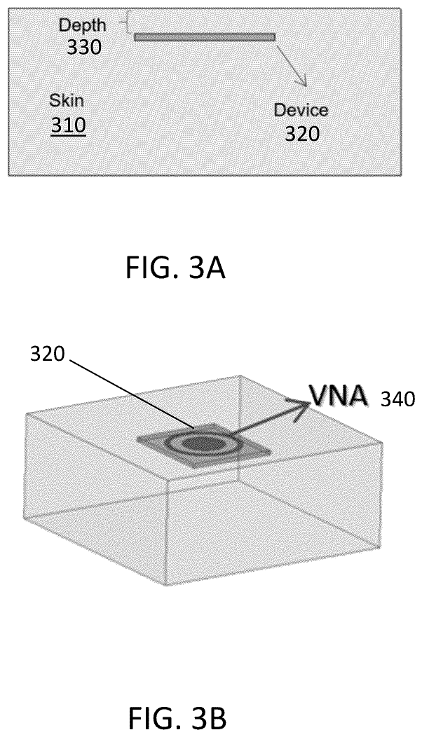

The antennas are fabricated on single-layer FR4 substrates, which have a dielectric constant of 4.4 and a thickness of 1.5 mm. A 50-Ω sub-miniature version A (SMA) adaptor connects the antenna to a vector network analyzer (VNA), as shown in FIGS. 3A and 3B. Herein, where a vector network analyzer (VNA) is disclosed, such disclosure is exemplary of any detector used for detecting a near-field resonance, e.g., a vector network analyzer, a scalar network analyzer, a spectrum analyzer, a phase-lock loop, or a frequency lock circuit. Embodiments of the invention may include any such detector used for detecting a near-field resonance. For finite-element simulations, the material permittivities for human skins are obtained from [29]. The permittivities and conductivities are frequency dependent. At 903 MHz, the relative permittivity and conductivity are 46.068 and 0.845 S/m, and at 2.45 GHz, 42.853 and 1.5919 S/m, respectively. The simulation configuration is shown in FIGS. 3A and 3B, with FIG. 3A showing skin 310 and the coupler 320 at a depth 330 in the skin 310, and FIG. 3B showing the coupler 320 connected to a VNA 340. The device is inserted into the skin with a variable depth. The design targets an implant depth of 6 mm at which the resonance occurs at 903 MHz or 2.45 GHz.

In measurements, the device is connected to a vector network analyzer (KEYSIGHT® PNA N5227B). Ground pork with about 27% of fat and 0.013 moles of salt in deionized water is used at room temperature as the phantom. The pork is packed in a cube of 100×100×50 cm³ and sealed with multiple layers of plastic wrap to keep moist with saline. As it is impossible to use human tissues for such experiments, certain disagreements on permittivity and conductivity are expected. Verification for the experiments is discussed below. Both the simulations and measurements are conducted up to 3 GHz, covering the two desired ISM bands for both the loop antennas with and without the center element.

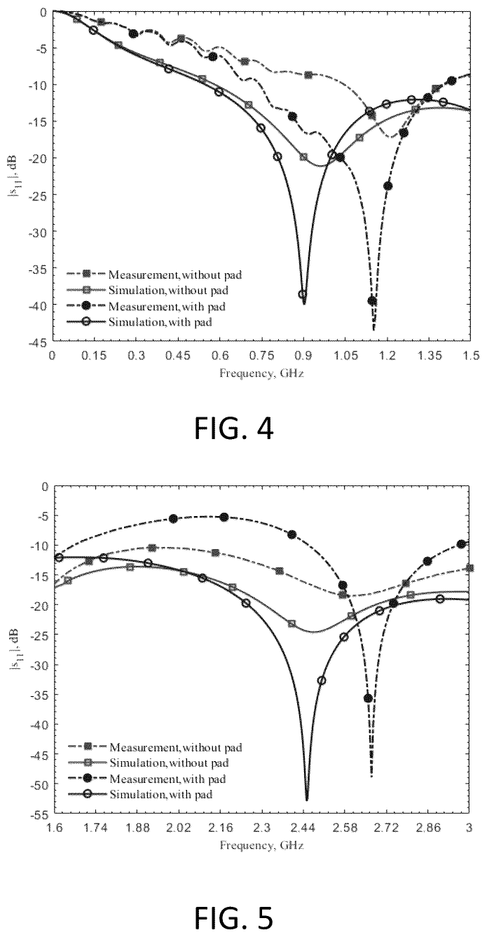

FIG. 4 shows the reflection coefficients for the 903-MHz antennas with and without the element when the implant is in a depth of 6 mm. Both simulation and measured results (blue curves) indicate better resonance, respectively, at 903 MHz and 1.152 GHz for the device with a center element. The gap d, in this case, is 4.5 mm. The case without the center element has its resonance at 960 MHz in simulation, although the loop is designed for 903 MHz according to the analytical equations [27]. This is due to the effective dielectric constant change around the antenna resulting from its implantation depth. The measured result shows a resonant frequency of 1.209 GHz. As expected, the permittivity of tissues and the depth change the resonant frequency. There is a 0.249-GHz frequency shift between simulation and measurement (dashed line). Both the cases with and without the center element have the same frequency shifts. This is due to the dielectric constant differences between human skin (simulation) and ground pork (experiment). This disagreement will be confirmed later. Comparing the measurements for the antennas with and without the center element, they clearly indicate a significant improvement in resonance with reflection coefficients from −17 to −43.5 dB. The figure data labels (squares and circles) are used to distinguish the curves. They are not the data points. Simulations are obtained with 801 points while measurements contain 1001 points. This applies to all the comparison figures herein. Comparing the measurement results with and without the center element, they clearly indicate that the improvement in resonance with a reflection coefficient from −17 to −43.54 dB using the test article of the embodiments of the present invention.

FIG. 5 shows the simulated and measured return losses for the resonating antennas with and without the element for the 2.4-2.5 GHz frequency band. The design targets the second resonance to be at 2.45 GHz. The element gap d is 1.5 mm. Similarly, the measured |s₁₁| is improved from −18.5 to −48.9 dB. A frequency shift of 0.219 GHz between simulation and measurement for the cases with the element is also observed. Although the case without the element design shows a resonance null, its measurement indicates a much less noticeable resonance due to the tissue permittivity. However, the cases with the element show clearly good impedance matching despite the pork tissue permittivity is different from that of skin. This highlights the need to have a robust resonance in the designs considering tissue property variations.

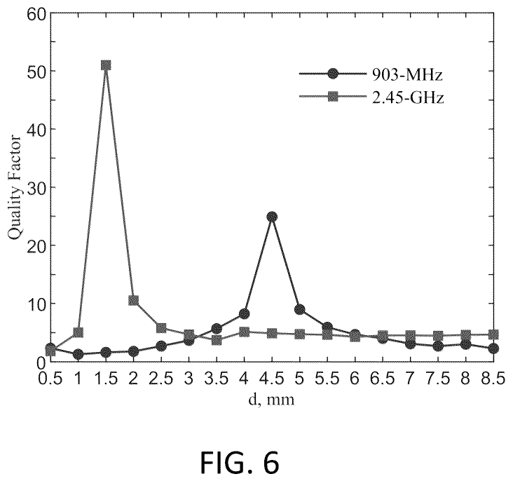

FIG. 6 shows the qualify factor comparison for the designs in two ISM bands. The quality factor is calculated from Q=f₀/Δf where f₀ is the resonant frequency and Δf is the 3-dB bandwidth. The quality factors reach maximum values of 24.91 and 51 with d=4.5 and 1.5 mm for the 903-MHz and 2.45-GHz designs, as compared to those of 2.12 and 4.80 for their counterparts of single loop designs without an element.

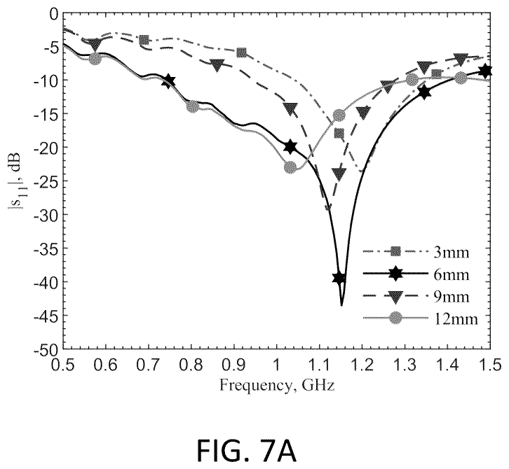

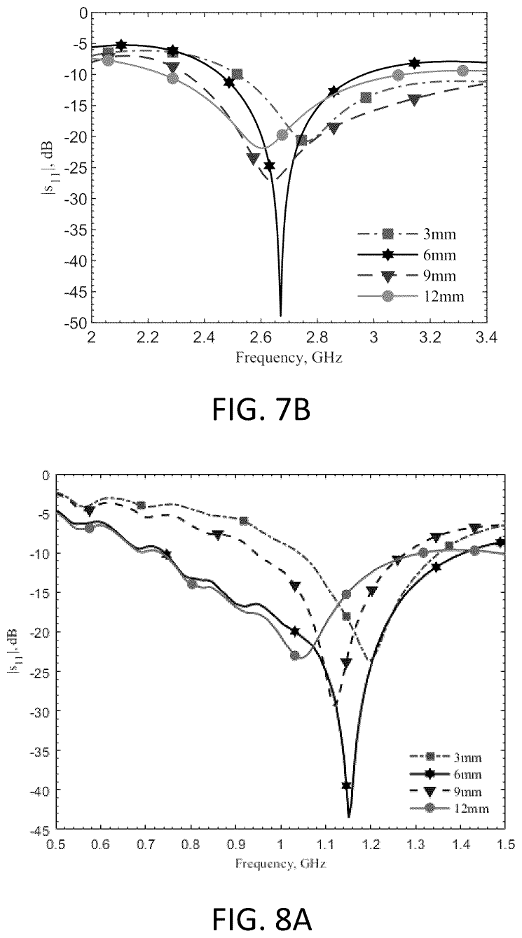

The implantation depth for these two ISM-frequency bands is designed at 6 mm. In realistic scenarios, surgeons may not have a way to control the depth so precisely. FIGS. 7A and 7B show the reflection coefficient changes at depths of 3, 6, 9, and 12 mm by simulations using human skin data, respectively, for the 903-MHz and 2.45-GHz designs. In both designs, the frequency shift percentages are within 6%, while the reflection coefficients and quality factors vary. The refection coefficients for all four cases are below −20 dB. In the cases without the tuning element, only the 6-mm cases have refection coefficients around −20 dB at the operating frequencies. This means the tuning element provides not only a better resonance at the operating frequency but also a more robust design against the depth variations.

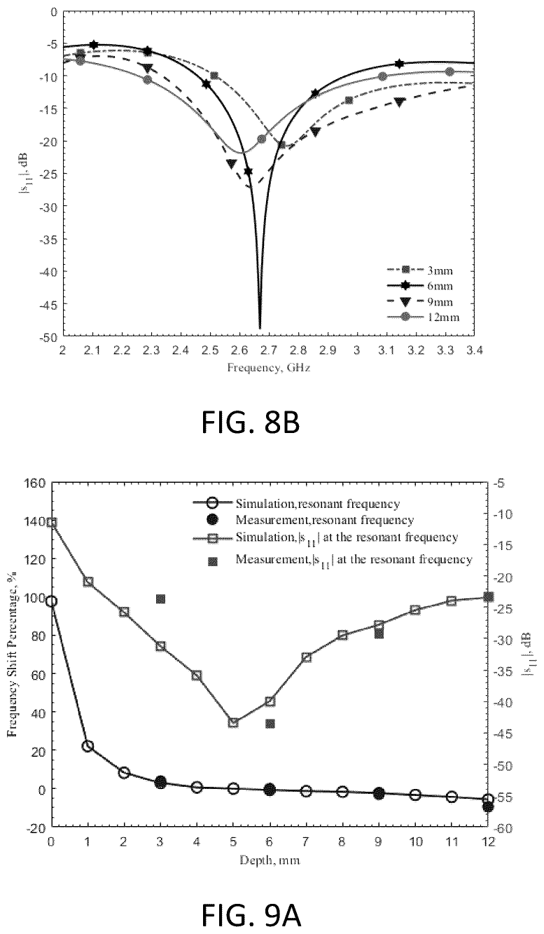

The inventors conduct experiments at different depths of 3, 6, 9, and 12 mm. The devices are inserted into a certain depth of the ground pork phantom. It is difficult to precisely control the varying depth steps less than 3 mm. FIGS. 8A and 8B show the measured resonances for the designs at 903 MHz (FIG. 8A) and 2.45 GHz (FIG. 8B). They match well with the simulations. Due to the permittivity of ground pork used, the measured resonant frequencies remain to have frequency shifts from the respective theoretical values utilizing the documented human skin permittivities. At the desired depth of 6 mm, the resonances are at 1.152 GHz and 2.67 GHz, respectively. Both plots show the same frequency shift trends at different depths. This is expected as the effective permittivity experienced by the antenna, which is affected by the field distribution proportions in air and inside tissues, changes resonance. The reflection coefficients are better than −20 dB in all cases.

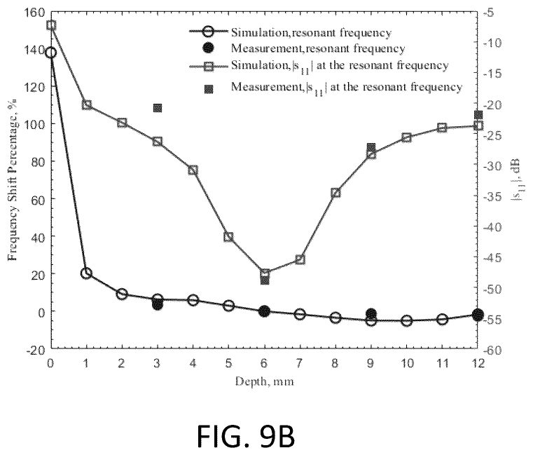

With the 6-mm depth designs for the resonant frequencies at 903 MHz and 2.45 GHz as the reference points and utilizing the documented frequency-dependent human skin permittivities and conductivities [29], simulations are conducted for various depths at a 1-mm step up to 12 mm. The resonant frequency shifts and reflection coefficients are shown in FIGS. 9A (903 MHz) and 9B (2.45 GHz). The measured results in FIGS. 9A and 9B are extracted, but with their resonances at 1.152 and 2.67 GHz, for the 6-mm depth, as the reference points. The measured resonant frequency shifts and return losses at the four discrete points of 3, 6, 9, and 12 mm match the theoretical results. For the depth of 3 mm, the frequency shifts match well, while the |s₁₁| has 7 and 5 dB discrepancies for 903-MHz and 2.45-GHz, respectively. Again, this is due to the differences of permittivities and conductivities in human skin dataset and ground pork. It should also be noted that the documented human skin permittivity data was obtained by measurements on skin, in which it combines all electromagnetic-wave effects from epidermis, dermis and fat layers as well as blood vessels and glands. The pork phantom is constructed with quasi-uniformly mixed fat and muscle tissues without layers. Thus, some differences between simulation and measurement are expected.

In conclusion, to maintain resonant frequency shifts less than 5% from the designed operating frequency, the implant depth should be deeper than 3 mm. Additionally, even if the transmitter can be dynamically tuned to reach the best resonance at the designed operating frequency, the implant depth should be kept between 4 and 8.5 mm to satisfy the requirement of reflection coefficient for the implant circuitry lower than −30 dB. If the requirement is at −20 dB, the implant depth can be at 2 mm to 12 mm.

There are disagreements between measurements and simulations due to the use of moist ground pork as phantom to mimic human tissues. The electrical parameters in simulations are obtained from [29]. For both 903-MHz and 2.45-GHz designs with/without the center element and at different implant depths, the frequency shifts between the theory and measurement are similar at 0.249 and 0.219 GHz, respectively.

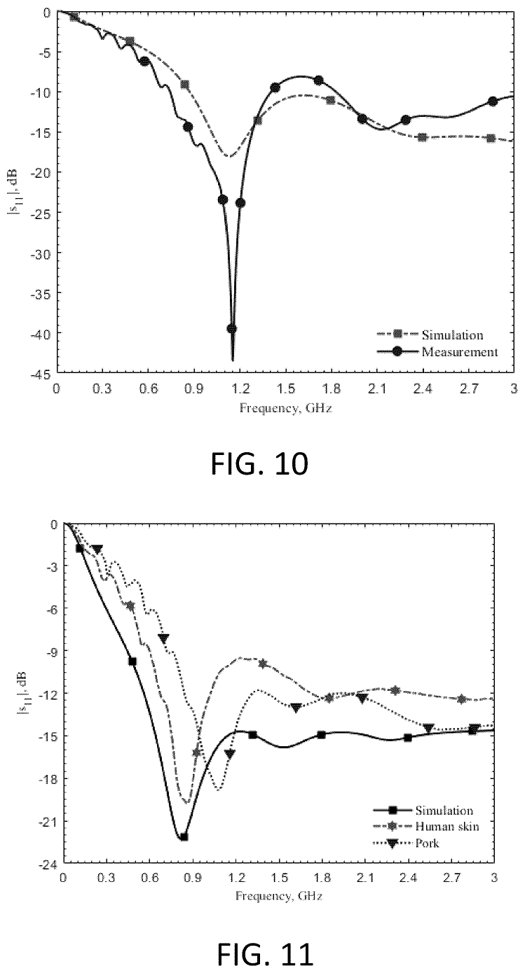

The inventors changed the permittivities and conductivities in the simulations to investigate the effects. With the human skin data from DC to 3 GHz as the base, uniform reductions on the relative permittivities and conductivity for all frequencies are used in a new phantom model to compare with the ground pork phantom. The rationale is that the pork contains more fat than human skin, so the permittivities should be lower at the frequencies of interest [30]-[32]. The pork does not contain interstitial fluid flows, so the conductivity should also be lower [33] [34]. For a relative permittivity decrease of 25 and a conductivity decrease of 0.35 S/m in the simulation, the result matches with the measurement at 1.152 GHz, as shown in FIG. 10.

For further validation, the inventors compare the scenario at the zero depth, by which the antenna is directly in contact with the skin. FIG. 11 shows the results of the simulation utilizing the human skin electrical properties (black curve), measurement with the antenna placed on the skin of a person's forearm (red), and on the top surface of the ground pork cube (blue). The resonant frequency in the simulation is 0.82 GHz, which matches the measured 0.86 GHz on the arm's skin, while the resonant frequency is 1.08 GHz for the ground pork phantom. The frequency difference of 0.22 GHz between the human skin and pork also agrees with those in other depths. From these two validation results, the designs utilizing the human skin properties could work well in realistic scenarios.

In summary, an element as a tuning structure in the center of a loop (split ring) antenna improved the resonance condition for wireless power and data transfer. The metal element in the center can also serve as the space to accommodate electronics including microprocessor, power management and sensor driver. Exemplary designs were demonstrated at two ISM bands with first and second resonant frequencies, respectively. The resonance for inductive coupling and thus impedance matching for the circuitry of embodiments of the present invention were shown to be greatly improved under practical constraints for subcutaneous implants.

The changes in resonant frequency and return loss due to implant depth deviation that occurs in the practical scenarios were also investigated. The tolerance for implantation depth was examined. Acceptable resonant frequency shift and reflection coefficient for the implant circuitry of embodiments of the present invention were shown to inform the desired implant depths.

Embodiments of the present invention may include a resonant coupler disposed on an attachment interface adapted to attach the resonant couple to an implant or a wearable device. The coupler resonance may be tuned by sizing one or more antenna or element dimensions by forming a selected gap width between a circumference of the element and the inner circumference of the loop. The coupler resonance may be tuned to maximize a quality factor of the coupler for a selected coupler reference frequency.

In embodiments of the present invention, the antenna and the element of the coupler may be adapted to be coupled to circuitry, adapted to receive or transmit electromagnetic energy to or from a surrounding environment, and adapted to transmit the electromagnetic energy to the circuitry. The circuitry may include a sensor, a stimulator, an energy storage device, or some combination. The surrounding environment may include an electromagnetic power source, and electromagnetic data transmitter, or an electromagnetic data receiver, or some combination.

An embodiment of a method of making a planar inductive resonant coupler may include sizing one or more dimensions of a circular planar loop antenna and an element, wherein the element is adapted to be disposed within and coplanar with a loop formed by the antenna; and disposing the element within the loop to form the gap.

An embodiment of a method for using a planar inductive resonant coupler may include providing a coupler including a circular planar loop antenna; and an element disposed within and co-planar with a loop formed by the antenna; wherein one or more antenna or element dimensions are sized to tune a coupler resonance of the coupler; disposing the coupler in an implant device or a wearable device; and sending electromagnetic energy to the coupler from an electromagnetic energy source or a data transmitter in a surrounding environment of the coupler, or receiving electromagnetic energy from the coupler with an electromagnetic power coupler or a data receiver in the surrounding environment of the coupler.

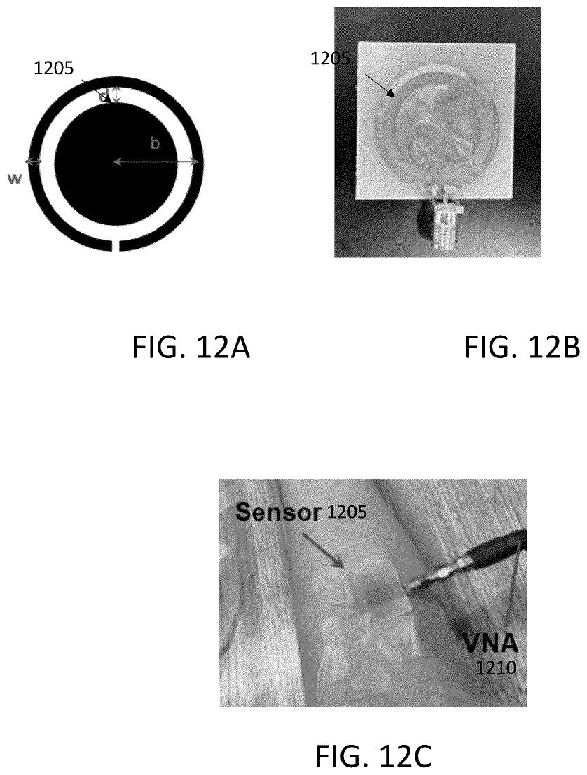

In this work, the inventors developed a near-field planar resonant loop as a wearable for noninvasive water content monitoring. The structure is based on a planar loop 1205 [53], with a metal pad embedded as a tuning element, as shown in FIG. 12A. The center pad provides distributed reactance to match the impedance of the loop at the desired resonant frequency. The inventors chose the ISM band around 912 MHz for these designs. Similar designs for improving power-transfer performance in subcutaneous implants have been investigated in the inventors' previous work [54, 55]. The results indicated that the planar tuned structure can be made in a compact size, yet with an excellent performance on resonance. Both are great features for wearable devices. Experiments and simulations were conducted to investigate the feasibility of monitoring human hydration. Additionally, general water content measurements for produce, like meat or fruit, were conducted to demonstrate wider applications of the sensor.

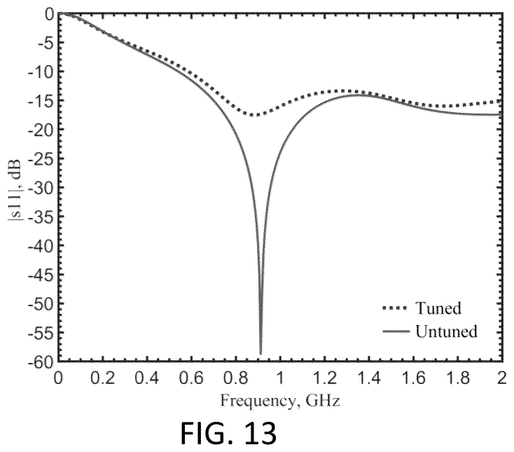

The sensor operates at around 912 MHz. It has a loop radius b=12 mm and a metal width w=1.6 mm. The spacing between the loop and pad is d=2.2 mm. The sensor 1205 is fabricated on a single-layer FR4 substrate with a dielectric constant of 4.4 and a thickness of 1.5 mm, as shown in FIG. 12B. Finite-element simulations are conducted with and without the metal center pad for comparison. The result is shown in FIG. 13. It clearly indicates a significant enhancement in resonance with the reflection coefficient |s₁₁| improved from −17.5 to −58.7 dB. The resonant frequency shifts from 882 to 912 MHz, which is acceptable. The material permittivities for human skin in simulations are obtained from the database in [54]. It should be noted that this database is generalized, and variations are expected for individuals. In the measurements, the sensor is placed on the person's forearm touching the skin. A 50-Ω SMA adaptor connects the sensor 1205 to a vector network analyzer 1210 (KEYSIGHT® PNA N5227B), as shown in FIG. 12C. Medical-grade tapes are used to fix the device in order to avoid movements. The participant stops taking in water or liquid after 10 PM on the night before the experiment. At 9 AM the next day, the participant completes jogging for 45 minutes on a treadmill prior to the measurements. After the sweat is wiped dry and the body temperature cools down to the one before jogging, the participant starts to continuously and slowly sip water. Reflection coefficients are recorded every 10 minutes up to 70 minutes. The final resonant frequency is considered as the fully hydrated state and is used as the baseline to calculate the frequency shift in percentage for other time points. The frequency in the beginning is treated as the initial dehydrated level, which depends on body conditions and is different on different days, even for the same participant who follows the same protocol. A total of seven datasets are collected in this study.

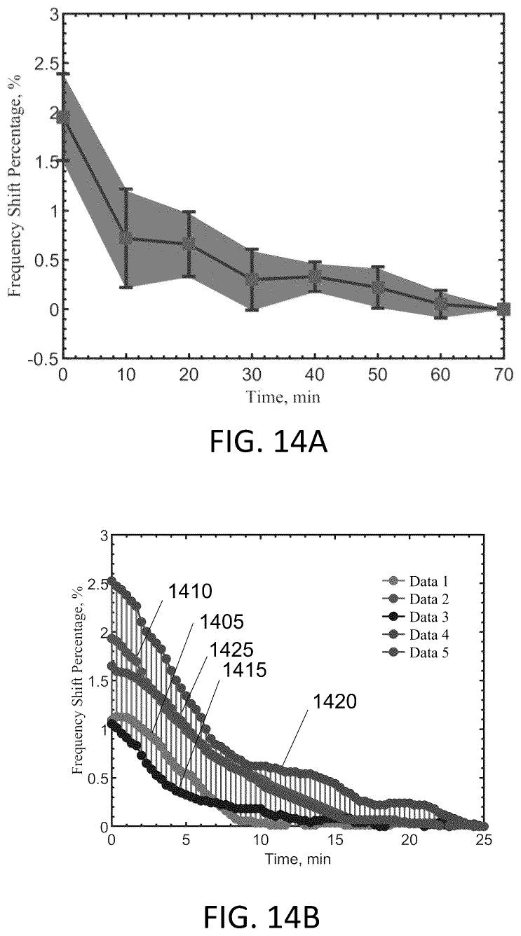

FIG. 14A shows the results of monitoring hydration progresses. Each dataset has a different initial dehydrated level, ranging from 1.4% to 2.4% at the start of the experiments, as expected. After the intake of water, gradual and monotonical descending trends are observed. The resonant frequencies stop shifting after 60 minutes, indicating the persons are fully hydrated.

FIG. 14B shows resonant frequency shifts in time during the human hydration experiments with five dehydrated participants. The data sets shown are data 1 (1405), data 2 (1410), data 3 (1415), data 4 (1420) and data 5 (1425). Resonance frequencies were tracked along with their reflection coefficients at an interval of 20 seconds after they started to drink water continuously. Each person was dehydrated before the time zero. The hydration process started when each person started sipping water continuously. The plateau in the end frequency means that the person is fully hydrated. The end frequency is used as the baseline, and the other frequencies are calculated as shift percentages using the formula ((f−f_{END})/f_{END})×100%.

The experiments indicate the feasibility of utilizing a planar resonator for water content measurements in tissues. However, the connectors and soldering parts make it uncomfortable to the person and difficult to make a constantly firm contact with the forearm during the entire experiment period. Consequently, there are deviations in measurements, clearly due to the tension from the coaxial cable, which causes gaps to appear between the device and skin. To overcome this issue, an improved sensor is built.

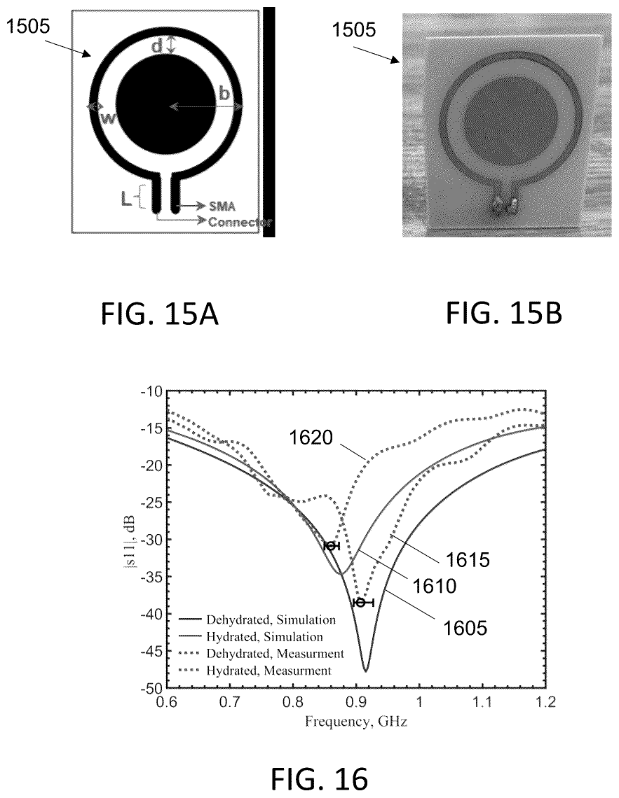

The sensor 1505, shown in FIGS. 15A and 15B, is modified with two extending legs from the loop gap in order to mount the SMA connector vertically. This configuration alleviates the cable tension. The loop is redesigned with a radius of loop b=13.6 mm, a metal width w of 1.6 mm, and a spacing between the metal loop and tuning pad d of 3.4 mm. The leg length L is 6 mm.

FIGS. 15A and 15B show its dimensions and a photo, respectively. Simulations are conducted using the documented dry and wet skin permittivities in [56]. The results, including measurements on the forearm, are shown in FIG. 16. The data sets shown in FIG. 16 are dehydrated, simulation (1605); hydrated, simulation (1610); dehydrated, measurement (1615); and hydrated, measurement (1620). In simulations, the dehydrated level at the resonant frequency of 915 MHz with |s₁₁| better than −47.8 dB. The resonant frequency shifts to 876 MHz for the case using the wet skin data, assuming to be the fully hydrated level, which is a 4.26% shift from 915 MHz. There are deviations in measurements due to the physiological condition of the person, the exact sensor location on the forearm, and the initial dehydrated levels in different experiments. The results show a dehydrated range of 849.7-872.7 MHz and a hydrated range of 896-927 MHz. The distinguishable frequency ranges indicate a robust performance.

The sensor is inserted into an adjustable compressing foam and then connected to the vector network analyzer. The forearm is comfortably confined by a layer of elastic foam to ensure a firm contact of the sensor on skin. The data is recorded every 20 seconds to provide more details on the hydration process. The reflection coefficient magnitudes are recorded as an indicator to monitor sensor movements. When its contact with skin changes, the reflection coefficient changes abruptly. The data are then filtered as body artifacts.

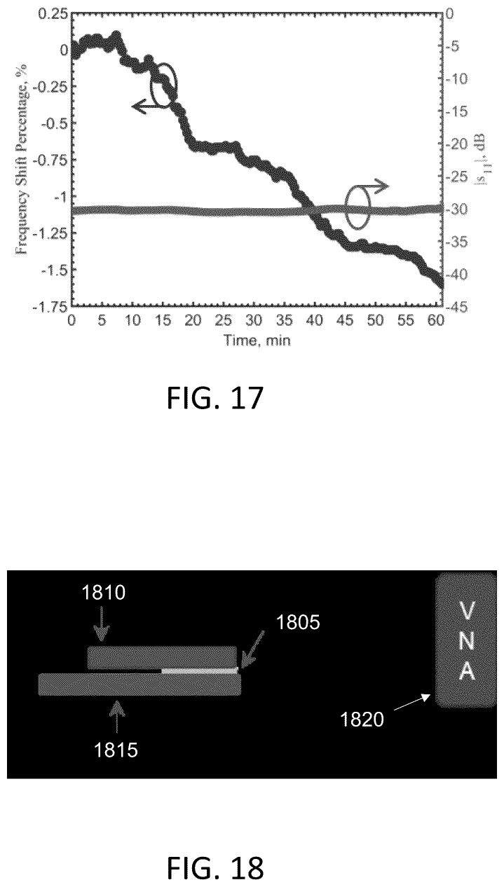

FIG. 17 shows the results from the higher sampling rate measurements and improved data processing method. Measurements are recorded every 20 seconds after the dehydrated person starts to continuously and slowly sip water every several minutes. The experiment lasts one hour. The baseline used is the resonant frequency at the beginning. As the person gets hydrated the frequency shifts a lower one and the shift percentage is calculated. The stable magnitudes of reflection coefficients indicate the frequency data without significant body artifacts. The declining trend in the resonant frequency shifts indicates the body is gradually getting hydrated during continuous water intakes. The results suggest that the sensor can detect the hydrating process in the human body continuously with a high temporal resolution.

The sensor also lends its features of being planar, noninvasive, and sensitive to water content to other applications, such as monitoring the water concentrations in produces like fruits or meats. Fresh orange and moist ground pork are used to demonstrate such an application.

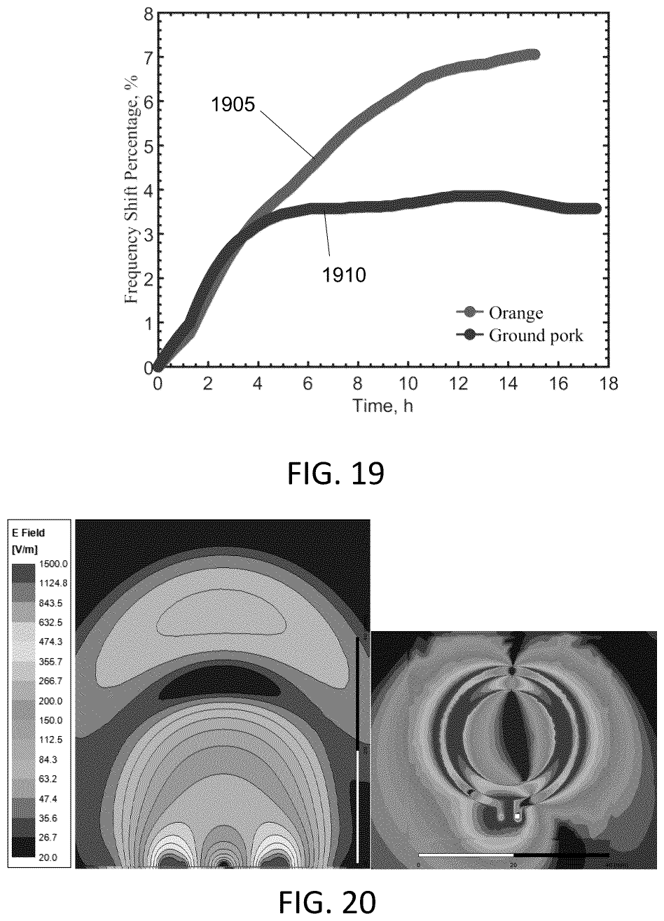

A slice of fresh orange is covered with a layer of plastic wrap. The wrap is open at the top of the slice to allow water to evaporate. The sensor 1805 is placed underneath the orange slice sample 1810 with porous styrofoam 1815, as shown in FIG. 18 with vector network analyzer (VNA) 1820. The experiment is designed this way because it is challenging to measure time-lapped water concentration changes with the skin remaining on the orange as it takes a long time for water to evaporate, even inside a dehydrator. Reflection coefficients are recorded every 3 minutes, and the total recording time is 15 hours. The start resonant frequency is served as the baseline to calculate the frequency-shift percentage. FIG. 19 (orange curve 1905) shows increasing frequency shifts as orange loses water. At the end of the experiment, there is a 7% frequency shift.

Similarly, another experiment is conducted using moist ground pork with the same setup as in FIG. 18. The ground pork is also sealed with a layer of plastic wrap with the top open for water evaporating. The sensor is placed underneath the patty, and measurements are taken every 3 minutes for 17.5 hours. A similar trend with the increasing resonant frequency is shown in FIG. 19 (blue curve 1910). The increasing trend slows down after 6 hours when the exposed surface of the tissues becomes tough and prevents water from going out. At the end of the experiment, there is a 3.5% frequency shift. Both experiments show that the sensor is sensitive to water content changes.

FIG. 20 shows how electromagnetic fields penetrate tissues. The sensor described herein is based on near-field resonance. The resonance does not depend on power so lower power, which can be lower than WiFi signals, can be used. The near field is confined in a limited region. In the simulation plots shown, it shows how deep the fields go into the body, estimated to be about 6-9 mm below the skin. The dermis layer under epidermis layer of skin is where the sweat glands and blood vessels (capillaries) are, so the fields are targeting the area that is affected by water the most. The fields do not travel through like waves so there is no worry about the power penetrating into deeper regions. The sensor discussed herein is not based on transmission or reflection measurements from one or more antennas. In those cases, the waves enter the tissues and interact with them. The waves get scattered in the tissues, so the transmission coefficient or reflection coefficient become different when the water in the tissues change them. This type of measurements rely on wave propagation in the tissues, so radiation power of the electromagnetic waves determine how deep the fields can penetrate. With different depths, (1) the scattering becomes different so the sensing performance becomes different; (2) there is a safety issue for tissues that the power can damage or heat up tissues; (3) the fields are not confined to a region, so it is difficult to predict how much radiation power is distributed among different types of tissues.

The inventors demonstrated a radio-frequency planar resonant loop for noninvasive water content monitoring. The sensor is susceptible to dielectric property changes due to water content variations. Human hydration experiments have been successfully conducted with discrete and continuous monitoring on the hydration processes. Both statistical experiments show distinct trends when a person gets hydrated from a dehydrated state. And it can be potentially made into a wearable with the advantages of being planar, compact, and with high sensitivity. Moreover, the sensor also has been used to identify water contents in produce. The demonstrations show great potential for a variety of noninvasive sensing applications in agriculture, the food industry, and diagnostic tools.

Any and all aspects of embodiments of the present invention disclosed herein are disclosed to be present together in any single embodiment unless prevented by physical impossibility.

As embodied and broadly described herein, an aspect of the present disclosure relates to a system for noninvasive monitoring of water content comprising, consisting essentially of, or consisting of: a radio-frequency (RF) planar resonant loop sensor comprising: a planar loop antenna; and an element disposed within and co-planar with a loop formed by the planar loop antenna; and a detector configured to be connected with the RF planar resonant loop sensor to detect a near-field resonance. In one aspect, the water is disposed within organic material comprising human tissue, non-human animal tissue, or plant tissue. In another aspect, the water is disposed within an inorganic material. In another aspect, the system is configured to be disposed on or about a surface. In another aspect, the detector comprises a vector network analyzer, a scalar network analyzer, a spectrum analyzer, a phase-lock loop, or a frequency lock circuit. In another aspect, the system is configured to measure an |s₁₁| reflection coefficient. In another aspect, the system is configured to monitor water content over time by measuring a resonance twice or more in a selected time period. In another aspect, the system is configured to monitor water content over time by measuring a resonance continuously during a selected time period.

As embodied and broadly described herein, an aspect of the present disclosure relates to a kit comprising, consisting essentially of, or consisting of: a radio-frequency (RF) planar resonant loop sensor comprising: a planar loop antenna; and an element disposed within and co-planar with a loop formed by the antenna; and a detector configured to be connected with the RF planar resonant loop sensor to detect a near-field resonance; and a device to secure the RF planar resonant loop sensor to a surface. In one aspect, the water is disposed within an organic material comprising human tissue, non-human animal tissue, or plant tissue. In another aspect, the water of is disposed within an inorganic material. In another aspect, the system is configured to be disposed on or about a surface. In another aspect, the detector comprises a vector network analyzer, a scalar network analyzer, a spectrum analyzer, a phase-lock loop, or a frequency lock circuit. In another aspect, the system is configured to measure an |s₁₁| reflection coefficient. In another aspect, the system is configured to monitor water content over time by measuring a resonance twice or more in a selected time period. In another aspect, the system is configured to monitor water content over time by measuring a resonance continuously during a selected time period.

As embodied and broadly described herein, an aspect of the present disclosure relates to a method of measuring water content in a material comprising, consisting essentially of, or consisting of: providing the material; providing a system for noninvasive monitor or water content comprising: a radio-frequency (RF) planar resonant loop sensor system comprising: a planar loop antenna; and an element disposed within and co-planar with a loop formed by the antenna; and a detector configured to be connected with the RF planar resonant loop sensor to detect a near-field resonance; disposing the loop on a surface of the material; and measuring a near-field resonance with the system. In one aspect, the material comprises an organic material comprising human tissue, non-human animal tissue, or plant tissue. In another aspect, the material comprises an inorganic material. In another aspect, the step of measuring the near-field resonance with the system comprises measuring an |s₁₁| reflection coefficient. In another aspect, the method further comprises measuring a near-field resonance twice or more in a selected time period. In another aspect, the method further comprises measuring a near-field resonance continuously during a selected time period.

Dehydration is a condition of lacking adequate water in the body. In general, more than 2% of body water loss can cause dehydration symptoms [57], leading to severe or even deadly health problems. For example, in a dehydrated state, the heart is forced to beat faster to compensate for the decrease in body blood circulation [58], potentially causing cardiovascular strain and damage to the heart and arteries [59], [60]. A well-hydrated state reduces the risk of heart failure, according to [61]. Dehydration also decreases the capacity to generate upper and lower body anaerobic muscular power, leading to a higher risk of musculoskeletal damage [62], [63], including muscle cramping and fatigue, which frequently occurs in marathons and triathlons [64], [65]. It has been well documented that dehydration can cause acute or chronic kidney injuries [66], [67], [68], [69], [70], [71], particularly they are important for certain groups of workers in the fields [72], [73] and urinary tract infections [74], [75], [76]. Seizures are also related to dehydration in patients with epilepsy and children [77], [78], [79], [80]. Apart from the physiological stress, dehydration also affects mental functions such as cognitive performance and mood [81] mainly due to shrunk blood vessels in the brain, and inefficient fluid supply [282]. Without normal water homeostasis, chronic neuronal dysfunction can lead to multiple neurodegenerative diseases, including dementia [83]. Therefore, maintaining a well-hydrated state is vitally important for body health.

The feedback mechanisms for a healthy person included in the urinary system and the sensation of thirst mainly regulate the balance of hydration by adjusting the urine volume [84] and intake volume of fluid inspired by the thirst feeling [85]. However, evaluating dehydration is challenging for people with diminished feedback mechanisms, including infants [86], [87], the elderly people [88], outdoor worker, athletes, and soldiers who are very concentrated on their tasks. Monitoring hydration levels becomes exceptionally vital for those people with a higher risk of dehydration. If detected early, severe situations or long-lasting consequences can be avoided as minor dehydration can be treated simply by fluid intake [89].

To access the feasibility of hydration level detection, a series of dehydration measurements have been conducted, which were based on detecting the variations in dielectric properties of measured tissues, largely determined by the water content of biological tissues with radio frequency (RF) and microwave signals [90], [91], [92]. Shahzad et al. [93] conducted ex vivo dielectric dehydration measurements using an open-ended coaxial probe from 500 MHz −20 GHz on the freshly excised rat liver samples. The dielectric parameters were recorded at different time points post-excision, and the measurements showed decreasing shifts of more than 25% in both the real and imaginary parts of complex permittivity during 3.5 hours after excision. Pollacco et al. [94] investigated the correlation between the dielectric properties of biological tissues and different hydration levels from the measurements conducted on rat muscle and fat tissues in vivo and ex vivo. Dielectric parameters were measured from 500 MHz to 50 GHz at the constant room temperature of 77° F., showing the muscle tissue with a higher percentage of water content, compared to the fat tissue, represented a higher dielectric constant and conductivity. Similar trends can be found and verified in the literature with different means of measurements [95], [96], [97], [98], indicating that the dielectric properties of biological tissues are mainly affected by water contents. It can be explained because of the high dielectric constant of water and the presence of free ions from water in tissues, which increase conductivity. As a result, dehydration thus leads to a decrease in dielectric properties. Based on this correlation, RF and microwave sensors have been designed and used for water content sensing [99] with three major principles: resonance, transmission, and reflection.

Resonance cavities can acquire frequency characteristics from interferences in the media, and dielectric properties of the media can be calculated from the spectral characteristics, typically assuming the media are homogenous and isotropic or with simple layer configurations if not homogenous. However, measurements using conventional resonance cavities are limited for targeted media with high dielectric losses [100]. The physical shapes of conventional high-Q (quality factor) cavity resonators [101], [102], [103] are constrained to be used on the human body as a wearable sensor for continuously monitoring. Split ring resonators have been used for probing tissue properties for the features of being planar and with higher sensitivities compared to the conventional loop resonators. Two split rings in a cavity placed on a person's abdomen to monitor blood glucose levels have been demonstrated with clinical evidence [104], [105]. One split ring closer to the skin detected the tissue changes while the other ring served to provide the reference frequency. Relative frequency shifts gave insights into blood glucose variations. The cavity was fairly large, so it was not convenient to wear for a long time. A planar split ring between a transmission line made on a rigid composite substrate (ROGERS 5880 PTFE) was used to monitor blood glucose [106]. The demonstration was performed on dead skin samples with controlled interstitial fluid mimicking different scenarios in tissues showing clear frequency shifts. However, the quality factor of the resonator was not mentioned. Similarly, a large array of split rings coupled to a transmission line on a rigid substrates (ROGERS DUROID® 6010) was made to evaluate dielectric properties on multiple points of a dead tissue sample [107]. A complementary split-ring was also proposed for sensing tissue types and demonstrated with dead animal tissue samples [108].

The transmission methods consist of transmitter and receiver antennas, by which electromagnetic fields go through and interact with the measured media. Garrett et al. designed a transmission sensor to measure human hydration levels. With the electromagnetic waves passing the forearm, the effective permittivity could be mathematically evaluated and classified into hydrated or dehydrated state groups [109], [110]. Later, the method was utilized to conduct human experiments on fasting participants [111]. However, such a system might be bulky, and this had a lower potential for wearable purposes. It was difficult for long-term continuous monitoring due to discomfort unless the antennas can be made planar with conformability. Under such constraints, participants in the transmission measurement were only recorded before and after excise indiscrete time points, by which other metabolism activities may raise the possibility of error in calculations. Besides, high field density may raise safety concerns as the transmitted powers, especially in local areas, may not be controlled well for different body types.

The reflection method has relatively simpler physical structures, which can be potentially designed as a wearable for long-term continuous monitoring. The inducing electromagnetic wave interacts with the measured media, and the reflection coefficient can be evaluated to distinguish the dielectric properties of the media. Typically, the reflection sensor requires a dynamic matching circuit to achieve better resonance performance for its transmission and reflection modes. The matching circuits are usually bulky and bring design constraints. Additionally, near-field characteristics, sizes, and wave scattering across different layers of tissues in reflection coefficient measurements make it challenging to design planar antennas with compact sizes as wearables to accomplish the required reliability and stability for different body types. Brendtke et al. [112]proposed a broad-band antenna at 7.9 GHz to measure the reflection coefficient on the skin equivalents that were made of cells and hydrogel with specific hydration and density of matrix components. Results indicated resonant frequencies and return losses were related to the corresponding skin equivalents and could be evaluated to determine hydration level provided by the designed skin equivalent. KilpijArvi et al. [113] designed a reflection sensor based on a complementary split-ring resonator (CSRR) pattern. The sensor is fabricated on an SMA connector that connected to a vector network analyzer (VNA), behaving as a dielectric probe. A series of different combinations of carbon black powders and barium strontium titanate were used with urethane rubber as skin phantoms to mimic the dielectric properties of dehydrated skins. The SMA connector formed a feeding structure to the CSRR. So the sensor may not be suitable for applications that require a planar and deformable configuration.

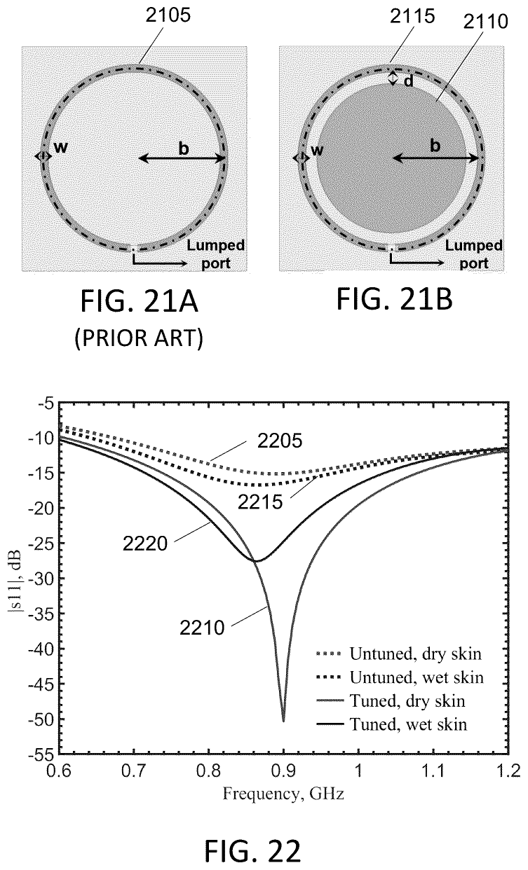

This work developed a flexible near-field planar resonant loop as a wearable for non-invasive water monitoring, in addressing the aforementioned constraints in different types of measurements methods and demonstrating in vivo on the human body. The design is based on a prior art planar loop 2105 [114], as shown in FIG. 21A, with a metal pad 2110 embedded within a planar loop 2115, as shown in FIG. 21B. The planar loops in FIG. 21A have been used widely for power coupling and sensing by static or RF magnetic fields. They provide simplicity and predictable characteristics in the air [114]. However, they have been limited by lower quality factors for resonance. The magnetic field sensing mainly depends on permeability instead of permittivity, to which the water content sensing effects are sensitive. The metal pad 2105 provides distributed capacitance [115] and mutual inductance [116] between the gap of the loop and the center pad in FIG. 21B. The distance of this gap serves to tune the distributed capacitance and sequentially the mutual inductance to match the impedance of the loop at the desired resonant frequency. Thus, the sensitivity to resonance is influenced by the permittivity in the near-field region. The 900-MHz ISM (Industrial, Scientific, and Medical) band is chosen for the demonstration in this work. However, the principle can be applied for any frequency band. The results showed that the planar tuned loop can be designed to a compact size with excellent performance in resonance. Both are essential features for an effective wearable device. Measurements and simulations were conducted to investigate the feasibility of monitoring human hydration levels. Moreover, general water content monitoring experiments were conducted with a phantom made of ground pork to show potential sensor applications.

Improvement of resonance and sensitivity. The proposed sensor was designed, shown in FIG. 21B, to conform onto the forearm skin and operate at around 900 MHz. The loop radius was b=11.75 mm with a metal width w=1 mm. The gap between the loop and the center pad was d=1.5 mm, which was optimized for the resonance on the human skin in the dehydrated condition. Considering the curvature of the forearm for a wearable, the tuned loop was fabricated on a flexible polyimide film (DUPONT™ PYRALUX® FR9220R) that can be firmly in contact with the skin. The copper pattern was etched after photolithography was applied with a photomask on the photoresist-covered film. A previous demonstration of the tuned loop for a wirelessly-powered implant application was conducted on rigid substrates [117]. The thickness of the film is 76 μm with a dielectric constant of 3.2. The copper thickness on the film is 70 μm. Finite-element simulations for the loop resonators with and without the tuning pad, on dry (dehydrated) and wet (dehydrated) skins, were conducted for comparison. The human skin dielectric properties were obtained from the documented library in [119]. It should be aware that this database does not define a quantitative level of hydration for dry or wet skins and is highly generalized, so discrepancies with measurements on individuals are expected.

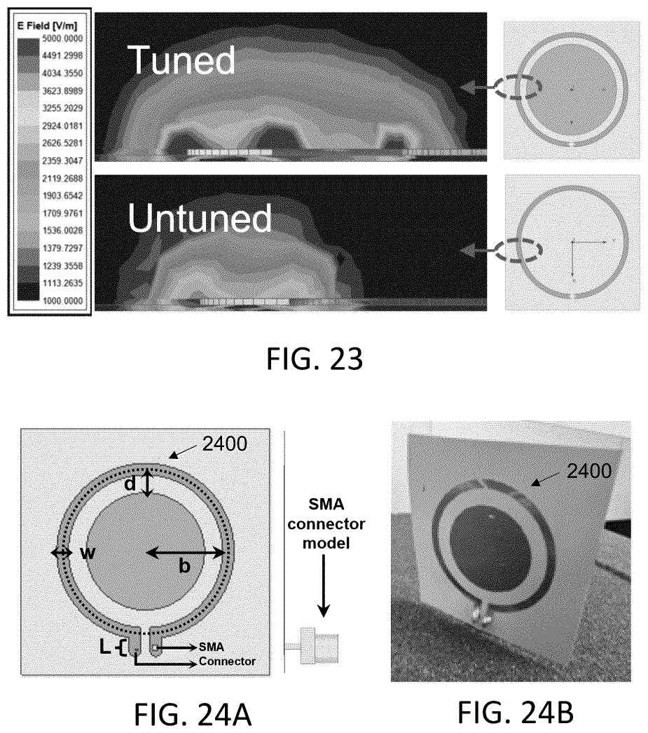

FIG. 22 shows the comparison of reflection coefficients. For dry skin, the single loop (dash curve 2205) has a resonant frequency at about 888 MHz with |s11| of −15.17 dB, whereas the sensor with the center pad (solid curve 2210) has a resonant frequency of 900 MHz with |s11| of −50.33 dB. FIG. 22 also shows data for the untuned single loop with wet skin, curve 2215, and for the tuned sensor with wet skin, curve 2220. The spectral shapes clearly indicate a significant enhancement in resonance with the |s11| improved by 35 dB. With the wet skin model, the loop experienced a frequency shift of 2%, while it was 4% for the tuned loop, as shown clearly in FIG. 22, from 900 to 864 MHz. The shift was distinct due to the better resonance that induced electric fields deeper into the tissues. FIG. 23 shows the higher intensity of electric fields in the tuned loop case, thus, the effective dielectric constant changes became more pronounced to affect the resonance.

Measurements. The inside of the forearm is selected for sensing. The skin is relatively thin for electric-field probing. The forearm area has close vicinity to the artery that delivers blood from the heart, it receives water from the bloodstream quicker than other body parts. The forearm provides access for a convenient and comfortable wearable. The sensor was placed on a person's forearm with medical-grade tapes (3M® NEXCARE® DURAPORE® Durable Cloth Tape, USA) fixed to avoid movement. A 50-Ω SMA adaptor connected the resonator to a vector network analyzer (KEYSIGHT® PNA N5227B). However, during the measurements, the connectors and soldering parts made the person feel uncomfortable and made it challenging to maintain firm contact with the skin. The horizontal connection from the leg and center conductor of the SMA connector experienced a contact issue between the substrate and the skin. Due to the mechanical tension from the rigid coaxial cable, coupled with the high sensitivity in resonance, measurements showed noticeable variations when applying different pressures on the connector. Similar phenomena in which probe contact pressures affected the RF measurements on tissues were observed [120]. To solve this issue, an improved sensor has been built.

The sensor 2400, shown in FIGS. 24A and 24B, was modified with two vias to mount the SMA adaptor vertically from the backside of the substrate. This allowed the sensor 2400 surface fully and firmly in contact with the skin. The contact between the SMA and the vias was not affected by the cable tension. Furthermore, the vertical cable arrangement permitted the cable to be mechanically supported by an elevated surface to ease tension. The loop was redesigned with a radius b=14.3 mm with a leg length L=3 mm. The spacing d between the loop and tuning pad was tuned at 3.1 mm. FIGS. 24A and 24B show its dimensions and a photo of the tuned loop sensor 2400 on a flexible polyimide substrate.

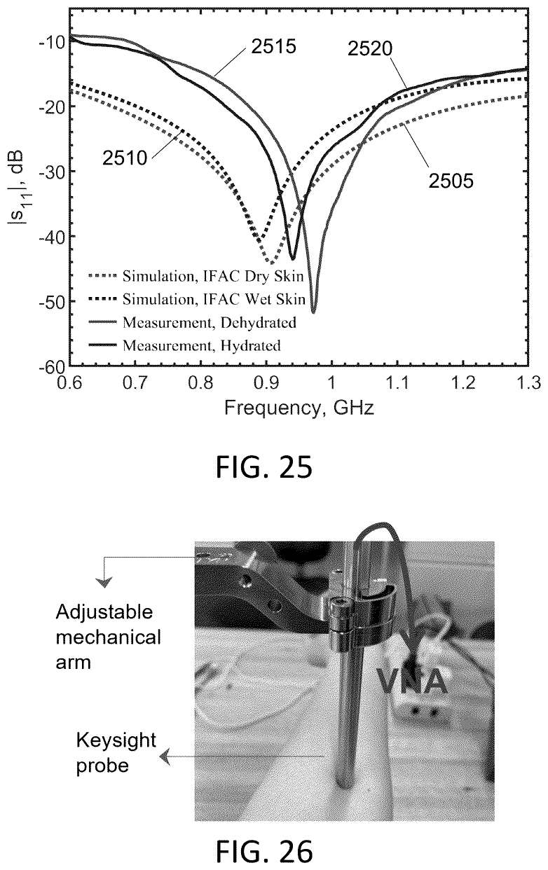

Simulations with the documented dry and wet skin permittivities [119] were conducted, and the results are shown in FIG. 25 with dry skin simulation results, curve 2505; wet skin simulation results, curve 2510; dehydrated measurement, curve 2515; and hydrated measurement, curve 2520. The sensor was tuned for the dry skin condition to operate at 906 MHz with a |s11| of −45 dB. The resonant frequency should shift to 888 MHz for the wet skin. Compared to simulations, the measured hydrated (wet) skin had a resonant frequency at 940 MHz while it was at 972 MHZ for the dehydrated (dry) skin. The creation of hydrated and dehydrated conditions in a person is discussed in a later section. The discrepancy in the resonant frequencies between the theoretical dry skin and the dehydrated person was 66 MHz. The discrepancy is 52 MHz for the theoretical wet skin and the hydrated person. The mismatch was due to the generalization of skin permittivities in the documented library used in simulations and that measurements for the individuals depended on the actual dehydrated levels at the time of the experiment.

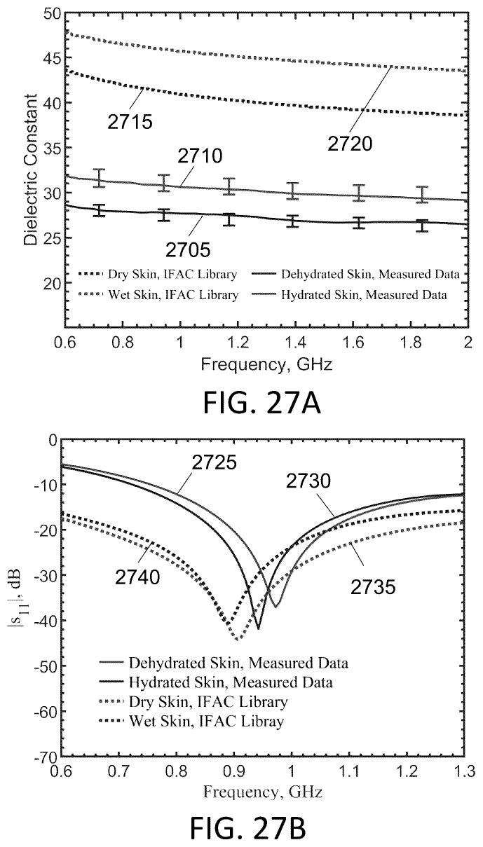

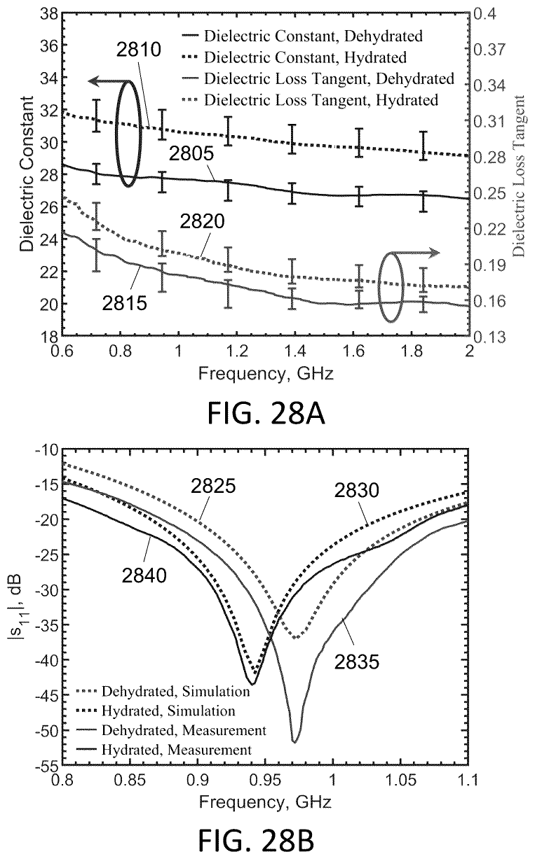

To verify the source of mismatch, dielectric properties measurements on the human forearm were conducted using a coaxial probe kit (KEYSIGHT® N1501 A). When the person was in the dehydrated or fully hydrated states, the probe was placed on the skin of the forearm, as shown in FIG. 26, and multiple dielectric properties measurements were repeated in the same location. The person stopped liquid and food intake after dinner and jogged for 45 minutes in the morning. After sweat was wiped and heart rate/body temperature returned to those before jogging, the probe was placed to measure the dielectric property of skin in the dehydrated state. Then the person started drinking water slowly and continuously for 30 minutes. After 30 minutes, if the reflection coefficients stayed unchanged, it was considered in a fully hydrated state. The measured dielectric constants (solid curves; dehydrated skin, measured data, curve 2705; hydrated skin, measured data, curve 2710) are shown in FIG. 27A, compared to those from the documented dataset [119] (dotted curves; dry skin, IFAC Library, curve 2715; wet skin, IFAC Library, curve 2720). The error bars show the ranges in 4 measurements. It was observed that pressure on the skins affected the measurement results, so the probe was pushed lightly on the skin, and multiple measurements were taken quickly. There were noticeable discrepancies in the skin dielectric properties between the generalized library dataset and the measurement from the individual person. This is expected because the datasets are generalized cases. The dehydrated (dry skin) levels also depended on individuals' physiological conditions. Using the measured dielectric constants in the models, FIG. 27B shows the simulations of reflection coefficients for the tuned loop resonator (solid curves; dehydrated skin, measured data, curve 2725; hydrated skin, measured data, curve 2730) compared to those using the documented library dataset (dotted curves; dry skin, IFAC Library, curve 2735; wet skin, IFAC Library, curve 2740). The discrepancy between using the measured skin data from a dehydrated individual and using the generalized skin data was 67 MHz. The discrepancy was 55 MHz for wet/hydrated conditions. FIGS. 27A and 27B highlight the mismatch between the generalized skin dielectric constants and the measured ones, as well as the effect on resonance. Thus, the inventors further considered including both the permittivity and conductivity of the dehydrated and fully hydrated tissues measured from human bodies in simulation. FIG. 28A shows the measured dielectric constants and loss tangents from 600 MHz to 2 GHz in one individual person (dielectric constant, dehydrated, curve 2805; dielectric constant, hydrated, curve 2810; dielectric loss tangent, dehydrated, curve 2815; dielectric loss tangent, hydrated, curve 2820). It should be noted that even with the same person, whose dehydrated and hydrated states should be the same, the measurements still contained deviations, as shown by the error bars in FIG. 28A. Utilizing the measured parameters in simulations, the resonant frequency for the dry skin was 972.5 MHz and shifted to 942.5 MHz for the wet skin. During the measurements for dielectric constants and loss tangents, the tuned loop resonator was also placed on the forearm near the probe, and the resonant frequencies were measured at 972 MHz and 940 MHz, respectively, for the dehydrated and fully hydrated states. The simulations and measurements match well, as shown in FIG. 28B (dehydrated, simulation, curve 2825; hydrated, simulation, curve 2830; dehydrated, measurement, curve 2835; hydrated, measurement, curve 2840). The resonance performances were robust for both the dry and wet skin conditions, with their reflection coefficients better than −37 dB.

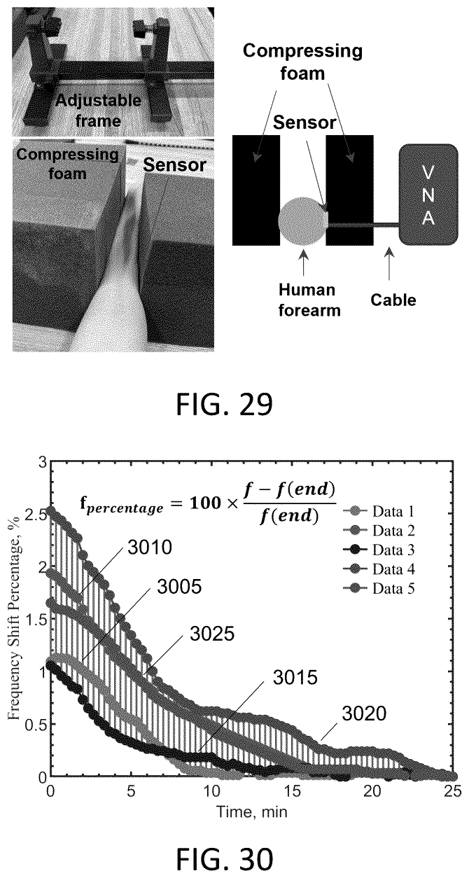

Experiment setup. The sensor was conformed onto the skin of the left forearm and connected to a vector network analyzer (KEYSIGHT® PNA N5227B). Considering different sizes in persons, a fixed measurement point became difficult. The measurements were conducted approximately 5 cm to the wrist on the forearm. Due to the high sensitivity of the tuned sensor and the mechanical tension from the coaxial cable, a small movement of the forearm caused measurement fluctuation, even with the vertically-mounted SMA adaptor. To further improve the stability, comfort was also important during the experiment since muscle fatigue occurrence was observed during experiments, and it induced involuntary motion of the arm. FIG. 29 shows the setup. An adjustable frame and elastic porous foams pressed the sensor firmly on the forearm. The compressing foam ensured by the adjustable frame comfortably conformed the sensor on the skin without feeling noticeable forces. The person could sit with the arm relaxed on the table during the long measurement period.

The experiments were designed to demonstrate the capability of continuously monitoring a person's hydration level in vivo. As it was difficult to create real-time and controlled scenarios of different human dehydration levels or have a means to independently and quantitatively verify the dehydration levels, the experiments aimed to investigate the measurements during the hydration process from a dehydrated state. The subject stopped the water, liquid, or food intake after 10 PM on the night before the experiment. At 9 AM the next day, the subject jogged for 45 minutes on a treadmill to induce sweating. After wiping out sweat, the subject rested until the skin became dry, heartbeat rates, and body temperature went back to normal. It was assumed that the subject was in a dehydrated state, verified by verbal acknowledgment of the person about feeling thirsty, although the dehydration level was not controllable or quantifiable because of variations in physiological conditions of individuals at different times. The human subjects research protocol ID is H21-023-CHIJ, approved on Apr. 19, 2021, by the Southern Methodist University IRB committee. Reflection coefficients were automatically recorded by the VNA every 20 seconds as soon as the subject started to sip water slowly and continuously while sitting without moving until 1000 mL of water was consumed. A program in MATLAB codes automatically sorted out resonance points in the frequency range of 0.1-2 GHz. It was found that the resonant frequencies in all cases plateaued within 25 minutes of hydration. The recorded resonant frequency at the fully hydrated state was used as the baseline, and frequency shifts in percentage were calculated for other resonant frequencies. A moving average of 10 samples was applied to all data to illustrate the trends.

Results and analysis. FIG. 30 shows the results of five cases (data 1, curve 3005; data 2, curve 3010; data 3, curve 3015; data 4, curve 3020; data 5, curve 3025). The initial dehydrated levels had frequency shifts from the baseline in percentage of 1.1%-2.5%, which depended on the individual physiological conditions. It was expected that it took more time to reach the fully hydrated state from a more serious dehydrated condition. The shaded area in FIG. 30 indicates the possible resonant frequency shift range. The gradual and monotonical trends showed clear evidence that the resonant frequency could indicate the body hydration level. The resonant frequency shifts changed faster in the first few minutes as the water entered blood vessels, which immediately altered the permittivities in the area electric fields reached. Simulations in later discussion showed the fields reached the dermis and hypodermis layers in the skin, which contain blood vessels and sweat glands. The trends gradually slowed down after a period of time. It might indicate that the water started entering cells until they were fully hydrated.

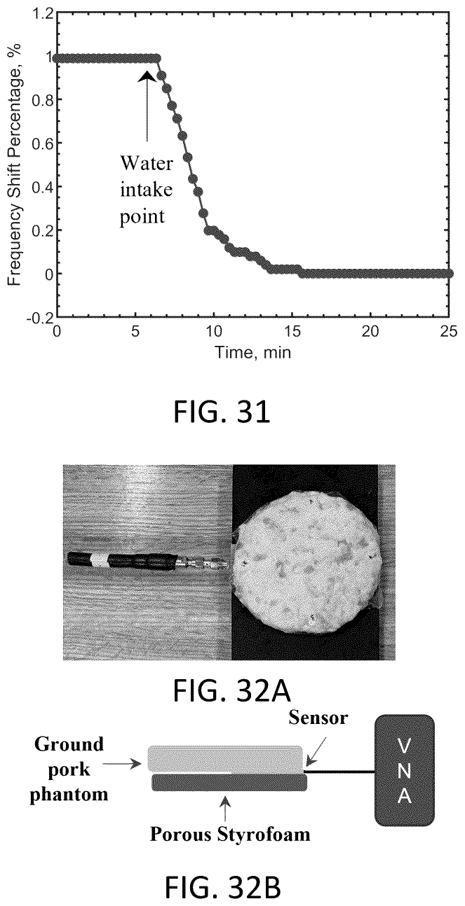

Another experiment for hydration monitoring was conducted. The experiment followed the same protocol except that the subject only drank 50 mL of water in the beginning, instead of a total of 1000 mL continuously. The recording began prior to the time point when the subject started drinking water. The baseline resonant frequency was the one after 25 minutes. FIG. 31 shows the resonant frequency shifts calculated from the baseline. The results revealed a monotonical descending trend after 50-mL water intake. The flat curve before the water intake indicated the measurement stability and that the resonant frequency shifts were solely caused by the hydration level changes. The total frequency shift was 1%, which was much lower than the average frequency shift in the other fully hydrated cases, indicating the subject was in a partial hydrated status by only taking 50 mL of water. The results verified the feasibility of using the tuned resonator with a robust resonance for continuously and noninvasively monitoring hydrating and dehydration levels in the human body.

A phantom to investigate water content. Specific hydration/dehydration conditions are difficult to create with good repeatability and control. The inventors created a phantom with moist ground pork for the intention to provide quantitative controls in experiments. The shape, thickness, weight, and added water volume were quantified to ensure experimental consistency. Proper amounts of water were mixed with ground pork to vary the water contents without water leaking to the bottom of the phantom. A 3D-printed cylinder disk with a height of 15 mm and a radius of 90 mm was used to pack ground pork. Fatty ground pork was chosen for its ability to better mix with water. Added water volume was 23 mL. After the ground pork was packed, it was removed from the disk and wrapped with a layer of plastic wrap. The phantom had the top surface exposed to air allowing water to evaporate, mimicking dehydration, as shown in FIG. 32A. The proteins, however, on the surface gradually became solidified when exposed to air for a long time, slowing down water evaporation. The weight of the ground pork phantom was 91 g.

FIG. 32B shows the setup for long-term water content monitoring. The tuned loop resonator was placed underneath the ground pork phantom. Porous Styrofoam was used to support the phantom and sensor while alleviating the cable tension. Reflection coefficients were recorded every minute for 19 hours. The resonant frequency at the beginning of the experiment was used as the baseline, and frequency shifts in percentage were calculated at other time points. The experiments were repeated with five phantoms under the same protocol. The weights of the phantoms were recorded at the end of the experiment, shown in Table 1. The weight loss percentages were treated as the water loss by evaporation.

TABLE 1
Weight Records of Moist Ground Pork Phantom

| | | | | Frequency |
|---|---|---|---|---|
| No. | Start Weight, g | End Weight, g | Weight loss, % | shift, % |
| 1 | 91 | 80 | 12.09 | 8.26 |
| 2 | 91 | 82 | 9.89 | 6.17 |
| 3 | 92 | 81 | 11.96 | 10.27 |
| 4 | 91 | 79 | 13.19 | 13.39 |
| 5 | 91 | 81 | 10.99 | 7.92 |

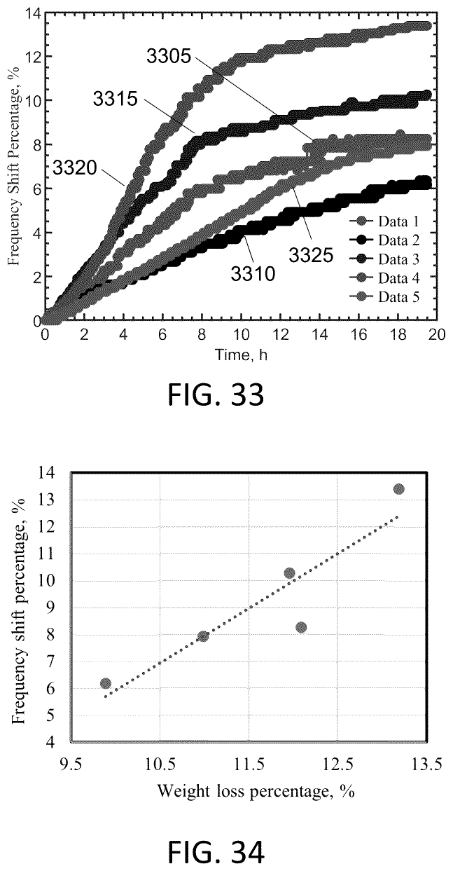

FIG. 33 shows the measured resonant frequency shifts in time during the 19-hour experiments. All five datasets (data 1, curve 3305; data 2, curve 3310; data 3, curve 3315; data 4, curve 3320; data 5, curve 3325) showed the continuously and monotonically increasing trends of resonant frequencies from the baseline during water evaporation. The trends slowed down during the second half of the experiments, mainly because the top part of the water in the phantom has evaporated, and the top surface became hardened, reducing evaporation rates.

The total frequency shifts were 8.26%, 6.17%, 10.27%, 13.39%, and 7.92% from the baseline for the five experiments, shown in Table. 1, while the weight losses in percentage were 12.09%, 9.89%, 11.96%, 13.19%, and 10.99%, respectively. FIG. 34 shows the relationship between the frequency shift and the total water loss. It indicated more water losses led to more resonant frequency shifts, as expected. However, there was an inconsistency when comparing datasets 1 and 3. The water loss percentage in weight in dataset 1 was 12.09%, with a smaller frequency shift of 8.26%, compared to the water loss of 11.96% with a frequency shift of 10.27% in dataset 3. It was hypothesized that this exception occurred because the ground pork phantom was not made uniformly across all areas with added water, and the resonator interacted with the tissues locally owing to confined field distributions. The localized resonance measurement then had a mismatch with the total weight change due to water evaporation.

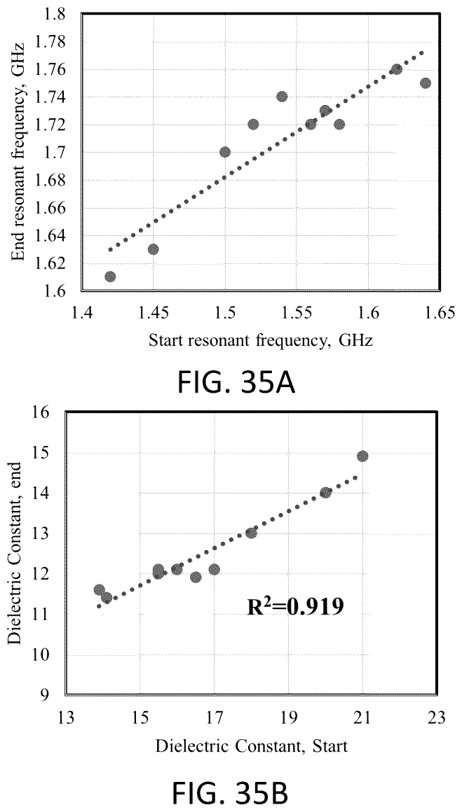

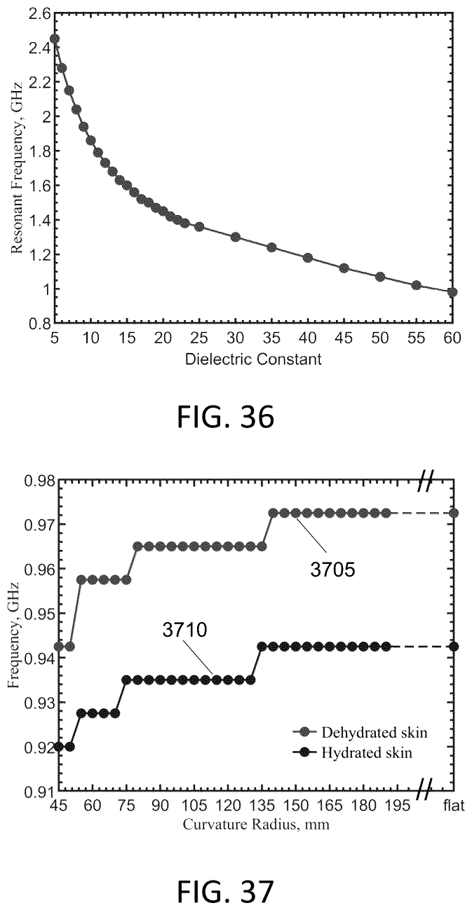

To validate the hypothesis, the same protocol was applied for ten measurements. Although the ground pork was mixed in a similar way and with the same weight and water amount, the resonant frequency at the start points of the experiments was different. The start and end resonant frequencies were recorded for each 19-hour experiment, as shown in FIG. 35A. A correlation was found between the start and end resonant frequencies after the water evaporation. Simulations were conducted in a model where the tuned loop resonator was placed on the boundary of air and a medium. The medium was assumed infinite in dimensions with a dielectric constant varied from 5 to 60. FIG. 36 shows the resonant frequency as a function of the dielectric constant. The effective permittivity changes, induced by the water content changes, then could be calculated from the resonant frequencies, as shown in FIG. 35B. The relationship with a linear regression R²=0.919 showed how the effective dielectric constant changed with the water losses. Even though the start resonant frequency was different due to local tissues, its changes and associated dielectric constant changes from the water losses followed the same trend. The dielectric constants calculated from the resonant frequencies are the mixtures of ground pork and added water, before and after evaporation. The permittivities of dielectric mixtures can be approximated by the two-phase Wiener mixing formula [121], [122]. With the dielectric constants 5.9 [123], [124], [125] and 80 of blood-infiltrated fat and water, the start and end dielectric constants were used to calculate the mixture ratios. As fat tissues induced weak depolarization fields by the external fields, if a depolarization factor of 0.333 was assumed for spherical distributions of induced charges, comparing the permittivities before and after evaporation, the ratios of water loss in total mixture volumes were between 10% and 15% for the cases in FIG. 35B.

Sensitivity on forearm curvatures. The sensor was designed and fabricated on a flexible polyimide substrate for wearables. Curvatures of forearms for different body types, especially children or infants with much smaller arms, needed to be considered. With a curvature, the resonant loop performance changed as the physical distributions of electromagnetic fields changed. Simulations were carried out by placing the resonator on curved arm models. Considering the shape of the sensing area in the forearm and various arm sizes, the curvature radius was estimated in a range of 45-190 mm to cover from infants to adults. The dehydrated and hydrated skin permittivity properties applied in the forearm model were measured by the dielectric probe (KEYSIGHT® N1501 A) on the human body following the protocol mentioned before.

FIG. 37 shows the resonant frequencies for hydrated skin (curve 3705) and dehydrated skin (curve 3710) in the selected curvature radius range from 45 to 190 mm. The circles indicate the calculated data. The resonant frequencies between hydrated and dehydrated states were distinguishable even with a small curvature radius. This is owing to the higher quality factor in resonance provided by the tuned loop resonator. The robust performance suggested that the sensor could be used in different body types.

Electric field distributions. Human skin consists of three layers: epidermis, dermis, and hypodermis layers with typical mean thicknesses of 0.075, 1.3, and 8 mm on the inside of the forearm [126], [127], [128]. Measurement of skin impedance at low frequencies to evaluate hydration is limited by the current and field distributions that are mainly on the surface of the epidermis layer [129]. To effectively measure hydration levels in the body, higher frequency signals allow the fields to reach deeper into tissues. However, measuring reflection and transmission coefficients with transmitting and receiving antennas to probe tissue properties requires higher powers, and the penetration of RF energy through the body often raises safety concerns. The applications of a resonator on the skin to probe a local area potentially resolve the concerns. The dermis layer is typically 1.3 mm below the surface and contains blood vessels, including capillary loops and sweat glands [130]. Blood vessels directly carry water while sweat glands, controlled by the sympathetic nervous system, secrete water. The dermis layer also contains elastin and collagen fibers, as well as fat cells and fibroblasts, which respond slower to water changes compared to blood vessels and sweat glands [131]. Therefore, the field coverage needs to be sufficiently deep in order to include the effects of the blood vessel and sweat glands on the effective permittivity changes from water.

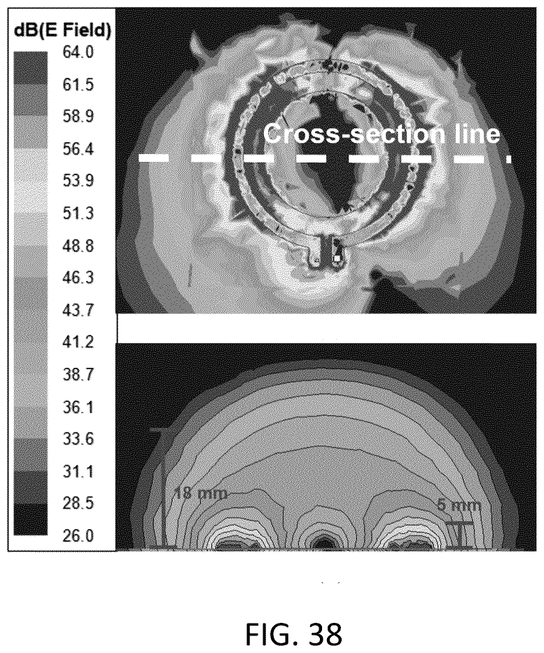

The tuned loop resonator is a near-field sensor since it is not effective for radiation with a tuning pad in the center. Simulations of field distributions were conducted. FIG. 38 shows the top and side views (upper and lower panels, respectively) of the electric fields at the resonant frequency of 900 MHz. The top view was taken in the plane of the tuned loop. The high electric fields were expected between the gap and around the outer ring. A standing wave was established for the tuned loop at the first resonance, so the fields had the lowest intensity between the gap in the middle of the loop, opposite to the port location. The side view revealed that the field magnitude decayed by 10 and 20 dB at depths of 5 and 18 mm, respectively, from the skin surface. The majority of the fields were confined in a cylinder with a diameter of 4 cm and a depth of 18 mm. Thus, it was assumed that the resonant frequencies were mainly determined by the effective permittivities in these specific areas. Given a sufficient field depth in the dermis and hypodermis layers to detect water influence in them, one does not need to worry about the energy penetrating too deep that it affects body functions or damages tissues.

It is demonstrated herein that a radio-frequency planar tuned loop resonator for non-invasive water content sensing within the body, potentially as a wearable on the forearm. Measurements and simulations were conducted to validate the feasibility. Discrepancies induced from the generalized tissue properties were verified. The high quality-factor of resonance in the tuned loop ensured the detection sensitivity for both the hydrated and dehydrated states. Utilizing measured effective permittivities in simulations verified the accuracy of using the planar resonator to detect hydration levels from a dehydrated state. Multiple experiments further demonstrated the feasibility of sensing hydration processes. A phantom was built to investigate the individuality of hydration levels in tissues and the resonance dependency on the tissue location.

The dehydration experiments conducted with the phantoms showed the relative changes in resonant frequencies can be related to the water losses, and the dependency was due to the local permittivities. The shifting of resonant frequencies induced by the curvature of the forearm, taking into consideration of different body types, did not present an issue as the resonant frequencies were distinguishable between hydrated and dehydrated states. The field distributions were investigated by finite-element simulations showing the fields were confined with a limited area yet sufficiently deep to exploit the advantages of probing blood vessels and sweat glands in the dermis and hypodermis layers of skin. The localized sensing relieved the exposure safety concern from electromagnetic energy.

The multiple experiments on human bodies showed distinct and repeatable trends from the individual dehydrated states to fully hydrated ones. For individual persons, calibration at the fully hydrated state will be needed, and the resonance can track continuously if the person experiences dehydration, particularly in exercise or training activities, or for the elderly and children to monitor their water intake. With the advantages of being planar, compact, flexible, and with high sensitivity, the tuned loop resonator can be used as the sensing element in a wearable with comfort. The demonstrations with phantoms also show great potential for a variety of non-invasive water-sensing applications, for example, detecting water percentages of fruits in agriculture, probing tissues or organs as diagnostic tools, and monitoring water retention or absorption in the food processing industry.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step, or a limitation) or group of integers (e.g., feature(s), element(s), characteristic(s), property(ies), method/process(s) steps, or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least 1, 2, 3, 4, 5, 6, 7, 10, 12 or 15%.

All of the devices and/or methods disclosed and claimed herein can be made and executed without undue experimentation in light of the present disclosure. While the devices and/or methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope, and concept of the invention as defined by the appended claims.

Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

To aid the Patent Office, and any readers of any patent issued on this application in interpreting the claims appended hereto, applicants wish to note that they do not intend any of the appended claims to invoke 35 U.S.C. § 112(f) as it exists on the date of filing hereof unless the words “means for” or “step for” are explicitly used in the particular claim.

### REFERENCES

1. Gonzalez, A.; Goikolea, E.; Barrena, J. A.; Mysyk, R. Review on Supercapacitors: Technologies and Materials. Renewable & Sustainable Energy Reviews 2016, 58, 1189-1206.
2. Poonam; Sharma, K.; Arora, A.; Tripathi, S. K. Review of Supercapacitors: Materials and Devices. Journal of Energy Storage 2019, 21, 801-825.
3. Deb, S.; Tang, S.; Abell, T. L.; McLawhorn, T.; Huang, W.; Lahr, C.; To, S. D. F.; Easter, J.; Chiao, J. Development of Innovative Techniques for the Endoscopic Implantation and Securing of a Novel, Wireless, Miniature Gastrostimulator (with videos). Gastrointestinal Endoscopy 2012, 76, 179-184.
4. Bose, P.; Khaleghi, A.; Albatat, M.; Bergsland, J.; Balasingham, I. RF Channel Modeling for Implant-to-Implant Communication and Implant to Subcutaneous Implant Communication for Future Leadless Cardiac Pacemakers. IEEE Transactions on Biomedical Engineering 2018, 65, 2798-2807.
5. Goroszeniuk, T.; Kothari, S.; Hamann, W. Subcutaneous Neuromodulating Implant Targeted at the Site of Pain. Regional Anesthesia and Pain Medicine 2006, 31, 168-171.
6. Yang, X.; Fu, T.; Kota, P. K.; Tjia, M.; Nguyen, C. M.; Chiao, J. Lactate Sensors on Flexible Substrates. Biosensors (Basel) 2016, 6, 48.
7. Souvik Dubey; Chiao, J. Power Transfer for a Flexible Gastric Stimulator, IEEE BioWireless Conference, Austin, TX, Jan. 24-27, 2016.
8. Neihart, N. M.; Harrison, R. R. Micropower Circuits for Bidirectional Wireless Telemetry in Neural Recording Applications. IEEE Transactions on Biomedical Engineering 2005, 52, 1950-1959.
9. Campi, T.; Cruciani, S.; Palandrani, F.; De Santis, V.; Hirata, A.; Feliziani, M. Wireless Power Transfer Charging System for AIMDs and Pacemakers. IEEE Transactions on Microwave Theory and Techniques 2016, 64, 633-642.
10. RamRakhyani, A. K.; Mirabbasi, S.; Mu Chiao Design and Optimization of Resonance-Based Efficient Wireless Power Delivery Systems for Biomedical Implants. IEEE Transactions on Biomedical Circuits and Systems 2011, 5, 48-63.
11. Deb, S.; Tang, S.; Abell, T. L.; Rao, S.; Huang, W.; To, S. D. F.; Lahr, C.; Chiao, J. An Endoscopic Wireless Gastrostimulator (with video). Gastrointestinal Endoscopy 2012, 75, 411-415.e1.
12. Rao, S.; Dubey, S.; Deb, S.; Hughes, Z.; Seo, Y.; Nguyen, M.; Tang, S.; Abell, T.; Lahr, C.; Chiao, J. Wireless 449 Gastric Stimulators. Texas Symposium on Wireless and Microwave Circuits and Systems 2014, 1-4.
13. Rao, S.; Chiao, J. Body Electric: Wireless Power Transfer for Implant Applications. IEEE Microwave Magazine 2015, 16, 54-64.
14. Davies, C.; Komoroski, C.; Roy, L. Evaluation of An Innovative Spinal Cord Stimulator Platform for The Treatment of Chronic Pain. Pain Management (London) 2018, 8, 167-174.
15. De Wachter, S.; Knowles, C. H.; Elterman, D. S.; Kennelly, M. J.; Lehur, P. A.; Matzel, K. E.; Engelberg, S.; Van Kerrebroeck, P. E. V New Technologies and Applications in Sacral Neuromodulation: An Update. Advances in Therapy 2020, 37, 637-643.
16. Clinical Trial: Neuspera's Implantable Sacral Nerve Stimulation System in Patients with Symptoms of Overactive Bladder. US Fed News Service, US State News 2020.
17. Lam, C. K.; Rosenow, J. M. Patient Perspectives on the Efficacy and Ergonomics of Rechargeable Spinal Cord Stimulators. Neuromodulation (Malden, Mass.) 2010, 13, 218-223.
18. Jia, F.; Hao, H.; Meng, F.; Guo, Y.; Zhang, S.; Zhang, J.; Li, L. Patient Perspectives on the Efficacy of A New Kind of Rechargeable Deep Brain Stimulatorsl. International Journal of Neuroscience 2016, 126, 996-1001.
19. Rosenberg, J. EonR battery Recharging: Preliminary Findings of Three Prospective, Multi-centered, Post-market Studies, 24^{th} Annual Meeting, 2008.
20. Hornberger, J.; Kumar, K.; Verhulst, E.; Clark, M. A.; Hernandez, J. Rechargeable Spinal Cord Stimulation Versus Nonrechargeable System for Patients With Failed Back Surgery Syndrome: A Cost-Consequences Analysis. The Clinical Journal of Pain 2008, 24, 244-252.
21. Abejon, D.; Vancamp, T.; Monzon, E. M. A Cost-Consequence Analysis Examining the Differences Between Non-Rechargeable and Rechargeable Systems. Anesthesiology and Pain Medicine 2020, 10, e100308.
22. Clingan, J. A.; Patel, A.; Maher, D. P. Survey of Spinal Cord Stimulation Hardware Currently Available for the Treatment of Chronic Pain in the United States. Front Pain Res. 1: 572907 2020.
23. Viswanath, O.; Urits, I.; Bouley, E.; Peck, J. M.; Thompson, W.; Kaye, A. D. Evolving Spinal Cord Stimulation Technologies and Clinical Implications in Chronic Pain Management. Current Pain and Headache Reports 2019, 23, 1-6.
24. Dawood, A.; Brown, J.; Sauret-Jackson, V.; Purkayastha, S. Optimization of Cone Beam CT Exposure for Pre-surgical Evaluation of the Implant Site. Dento-maxillo-facial Radiology 2012, 41, 70-74.
25. Liu, J. J.; Lutkin, J. E. Imaging of Patients Having Metal Implant Using X-ray Computed Tomography. Journal of X-ray Science and Technology 2009, 17, 355-365.
26. Storer, J. E. Impedance of Thin-wire Loop Antennas. Transactions of the American Institute of Electrical Engineers. Part 1. Communication and electronics 1956, 75, 606-619.
27. McKinley, A. F.; White, T. P.; Maksymov, I. S.; Catchpole, K. R. The Analytical Basis for the Resonances and Anti-resonances of Loop Antennas and Meta-material Ring Resonators. Journal of Applied Physics 2012, 112, 94911.
28. Bing, S., Chawang, K. and Chiao, J. C., Resonant Coupler Designs for Subcutaneous Implants. Proceedings of the 2021 IEEE Wireless Power Transfer Conference, Wireless Power Week. Jun. 1-4, 2021. pp. 1-4.
29. Andreuccetti, D. et al. An internet Resource for the Calculation of the Dielectric Properties of Body Tissues in the Frequency Range 10 Hz-100 GHz, Institute of Applied Physics “Nello Carrara” (IFAC), CNR, 1997. Available: niremf.ifac.cnr.it/tiss prop/.
30. Gunasekaran, N.; Mallikarjunan, P.; Eifert, J.; Sumner, S. Effect of Fat Content and Temperature on Dielectric Properties of Ground Beef. Transactions of the ASAE, 48, 673-680.
31. Lyng, J. G.; Zhang, L.; Brunton, N. P. A Survey of the Dielectric Properties of Meats and Ingredients Used in Meat Product Manufacture. Meat Science 2005, 69, 589-602.
32. Ng, S. K.; Gibson, A.; Parkinson, G.; Haigh, A.; Ainsworth, P.; Plunkett, A. Bimodal Method of Determining Fat and Salt Content in Beef Products by Microwave Techniques. IEEE Transactions on Instrumentation and Measurement 2009, 58, 3778-3787.
33. van der Sman, R. G. M Model for Electrical Conductivity of Muscle Meat During Ohmic Heating. Journal of Food Engineering 2017, 208, 37-47.
34. Shirsat, N.; Lyng, J. G.; Brunton, N. P.; McKenna, B. Ohmic Processing: Electrical Conductivities of Pork Cuts. Meat Science 2004, 67, 507-514.
35. E. Jequier and F. Constant, “Water as an essential nutrient: the physiological basis of hydration,” European Journal of Clinical Nutrition, vol. 64, pp. 115-123.
36. L. Jones, M. Cleary, R. Lopez, R. Zuri and R. Lopez, “Active Dehydration Impairs Upper and Lower Body Anaerobic Muscular Power,” Journal of Strength and Conditioning Research, vol. 22, pp. 455-463, March 2008.
37. K. Watanabe, E. J. Stöhr, K. Akiyama, S. Watanabe and J. Gonzilez-Alonso, “Dehydration reduces stroke volume and cardiac output during exercise because of impaired cardiac filling and venous return, not left ventricular function,” Physiological Reports, vol. 8, pp. e14433-n/a, June 2020.
38. N. A. Masento, M. Golightly, D. T. Field, L. T. Butler and C. M. van Reekum, “Effects of hydration status on cognitive performance and mood,” British Journal of Nutrition, vol. 111, pp. 1841-1852, May 28, 2014.
39. K. E. D'anci, A. Vibhakar, J. H. Kanter, C. R. Mahoney and H. A. Taylor, “Voluntary dehydration and cognitive performance in trained college athletes,” Percept Mot Skills, vol. 109, pp. 251-269, —August 2009.
40. L. E. Armstrong, M. S. Ganio, D. J. Casa, E. C. Lee, B. P. McDermott, J. F. Klau, L. Jimenez, L. Le Bellego, E. Chevillotte and H. R. Lieberman, “Mild dehydration affects mood in healthy young women,” J Nutr, vol. 142, pp. 382-388, —February 2012.
41. G. Szinnai, H. Schachinger, M. J. Arnaud, L. Linder and U. Keller, “Effect of water deprivation on cognitive-motor performance in healthy men and women,” Am J Physiol Regul Integr Comp Physiol, vol. 289, pp. 275, —July 2005.
42. M. S. Ganio, L. E. Armstrong, D. J. Casa, B. P. McDermott, E. C. Lee, L. M. Yamamoto, S. Marzano, R. M. Lopez, L. Jimenez, L. Le Bellego, E. Chevillotte and H. R. Lieberman, “Mild dehydration impairs cognitive performance and mood of men,” Br J Nutr, vol. 106, pp. 1535-1543, —November 2011.
43. S. M. Shirreffs, S. J. Merson, S. M. Fraser and D. T. Archer, “The effects of fluid restriction on hydration status and subjective feelings in man,” Br J Nutr, vol. 91, pp. 951-958, —June 2004.
44. N. Pross, A. Demazieres, N. Girard, R. Barnouin, F. Santoro, E. Chevillotte, A. Klein and L. Le Bellego, “Influence of progressive fluid restriction on mood and physiological markers of dehydration in women,” Br J Nutr, vol. 109, pp. 313-321, —Jan. 28, 2013.
45. W. F. Clark, J. M. Sontrop, J. J. Macnab, R. S. Suri, L. Moist, M. Salvadori and A. X. Garg, “Urine Volume and Change in Estimated GFR in a Community-Based Cohort Study,” Clinical Journal of the American Society of Nephrology, vol. 6, pp. 2634-2641, Nov. 1, 2011.
46. B. M. Popkin, K. E. D'Anci and I. H. Rosenberg, “Water, hydration, and health,” Nutrition Reviews, vol. 68, pp. 439-458, August 2010.
47. K. E. D'Anci, F. Constant and I. H. Rosenberg, “Hydration and Cognitive Function in Children,” Nutrition Reviews, vol. 64, pp. 457-464, Oct. 1, 2006.
48. M. H. Gorelick, K. N. Shaw and K. O. Murphy, “Validity and Reliability of Clinical Signs in the Diagnosis of Dehydration in Children,” Pediatrics, vol. 99, pp. e6-e6, May 1, 1997.
49. P. A. Phillips, B. J. Rolls, J. G. G. Ledingham, M. L. Forsling, J. J. Morton, M. J. Crowe and L. Wollner, “Reduced Thirst after Water Deprivation in Healthy Elderly Men,” New England Journal of Medicine, vol. 311, pp. 753-759, Sep. 20, 1984.
50. Ebbe Nyfors, “Industrial Microwave Sensors—A Review,” Sensing and Imaging, vol. 1, pp. 23, Jan. 1, 2000.
51. G. S. Raghavan and M. S. Venkatesh, “An overview of dielectric properties measuring techniques,” Canadian Biosystems Engineering, vol. 47, pp. 7, Jan. 1, 2005.
52. D. C. Garrett and E. C. Fear, “Feasibility Study of Hydration Monitoring Using Microwaves-Part 1: A Model of Microwave Property Changes With Dehydration,” Jerm, vol. 3, pp. 292-299, December 2019.
53. A. F. McKinley, T. P. White, I. S. Maksymov and K. R. Catchpole, “The Analytical Basis for the Resonances and Anti-resonances of Loop Antennas and Meta-material Ring Resonators,” Journal of Applied Physics, vol. 112, pp. 94911, November 2012.
54. S. Bing, K. Chawang and J.-C. Chiao, “A Resonant Coupler for Subcutaneous Implant,” Sensors (Basel, Switzerland), vol. 21, pp. 8141, Dec. 6, 2021.
55. S. Bing, K. Chawang and J.-C. Chiao, “Resonant Coupler Designs for Subcutaneous Implants,” pp. 1-4, Jun. 1, 2021.
56. D. Andreuccetti et al. “An internet Resource for the Calculation of the Dielectric Properties of Body Tissues in the Frequency Range 10 Hz-100 GHz,” IFAC-CNR, 1997.
57. S. N. Cheuvront and R. W. Kenefick, “Dehydration: Physiology, assessment, and performance effects,” Comprehensive Physiol., vol. 4, pp. 257-285, January 2014.
58. K. Watanabe, E. J. Stöhr, K. Akiyama, S. Watanabe, and J. GonzálezAlonso, “Dehydration reduces stroke volume and cardiac output during exercise because of impaired cardiac filling and venous return, not left ventricular function,” Physiol. Rep., vol. 8, no. 11, June 2020, Art. no. e14433.
59. J. C. Watso and W. B. Farquhar, “Hydration status and cardiovascular function,” Nutrients, vol. 11, no. 8, August 2019, Art. no. 1866.
60. G. Arnaoutis et al., “The effect of hypohydration on endothelial function in young healthy adults,” Eur. J. Nutr., vol. 56, no. 3, pp. 1211-1217, April 2017.
61. N. I. Dmitrieva, D. Liu, and M. Boehm, “Increased risk of heart failure is associated with chronic habitual hypohydration that elevates serum sodium above 142 mmol/l suggesting lifelong optimal hydration as preventive measure,” Eur. Heart J., vol. 42, no. Supplement_1, October 2021, Art. no. e053141.
62. L. Jones, M. Cleary, R. Lopez, R. Zuri, and R. Lopez, “Active dehydration impairs upper and lower body anaerobic muscular power,” J. Strength Conditioning Res., vol. 22, no. 2, pp. 455-463, March 2008.
63. J. A. Kraft, J. M. Green, P. A. Bishop, M. T. Richardson, Y. H. Neggers, and J. D. Leeper, “The influence of hydration on anaerobic performance,” Res. Quart. Exercise Sport, vol. 83, no. 2, pp. 282-292, June 2012.
64. I. Martinez-Navarro, A. Montoya-Vieco, E. Collado, B. Hernando, N. Panizo, and C. Hernando, “Muscle cramping in the marathon: Dehydration and electrolyte depletion vs. muscle damage,” J. Strength Conditioning Res., vol. 36, no. 6, pp. 1629-1635, August 2020.
65. J. D. Coso et al., “Muscle damage and its relationship with muscle fatigue during a half-iron triathlon,” PLoS ONE, vol. 7, no. 8, 2012, Art. no. e43280.
66. C. Roncal-Jimenez, M. A. Lanaspa, T. Jensen, L. G. Sanchez-Lozada, and R. J. Johnson, “Mechanisms by which dehydration may lead to chronic kidney disease,” Ann. Nutr. Metab., vol. 66, no. 3, pp. 10-13, January 2015.
67. G. Strippoli, J. Craig, E. Rochtchina, V. Flood, J. Wang, and P. Mitchell, “Fluid and nutrient intake and risk of chronic kidney disease,” Nephrology, vol. 16, no. 3, pp. 326-334, March 2011.
68. J. M. Sontrop et al., “Association between water intake, chronic kidney disease, and cardiovascular disease: A cross-sectional analysis of NHANES data,” Amer. J. Nephrol., vol. 37, no. 5, pp. 434-442, May 2013.
69. C. Bongers et al., “Impact of acute versus prolonged exercise and dehydration on kidney function and injury,” Physiol. Rep., vol. 6, no. 11, 2018, Art. no. e13734.
70. C. L. Chapman, B. D. Johnson, N. T. Vargas, D. Hostler, M. D. Parker, and Z. J. Schlader, “Both hyperthermia and dehydration during physical work in the heat contribute to the risk of acute kidney injury,” J. Appl. Physiol., vol. 128, no. 4, pp. 715-728, April 2020.
71. J. A. Lo et al., “Impact of water consumption on renal function in the general population: A cross-sectional analysis of KNHANES data (2008-2017),” Clin. Erp. Nephrol., vol. 25, no. 4, pp. 376-384, January 2021.
72. D. H. Wegman et al., “Intervention to diminish dehydration and kidney damage among sugarcane workers,” Scand. J. Work, Environ. Health, vol. 44, no. 1, pp. 16-24, January 2018.
73. M. C. Houser et al., “Inflammation-related factors identified as biomarkers of dehydration and subsequent acute kidney injury in agricultural workers,” Biol. Res. Nurs., vol. 23, no. 4, pp. 676-688, October 2021.
74. S. Ninan, C. Walton, and G. Barlow, “Investigation of suspected urinary tract infection in older people,” BMJ. Brit. Med. J., vol. 349, no. 7972, pg. 4070, August 2014.
75. M. Ramzan, S. Bakhsh, A. Salam, G. M. Khan, and G. Mustafa, “Risk factors in urinary tract infection,” Gomal J. Med. Sci., vol. 2, no. 1, pp. 11-21, June 2004.
76. F. Manz and A. Wentz, “The importance of good hydration for the prevention of chronic diseases,” Nutr. Rev., vol. 63, no. Supplement_1, pp. 2-5, June 2005.
77. I. McQuarrie, “Epilepsy in children: The relationship of water balance to the occurrence of seizures,” Amer. J. Dis. Child., vol. 38, no. 3, pp. 451-467, September 1929.
78. R. D. Andrew, “Seizure and acute osmotic change: Clinical and neurophysiological aspects,” J. Neurological Sci., vol. 101, no. 1, pp. 7-18, 1991.
79. R. D. Andrew, M. Fagan, B. A. Ballyk, and A. S. Rosen, “Seizure susceptibility and the osmotic state,” Brain Res., vol. 498, no. 1, pp. 175-180, 1989.
80. A. K. Doumbia et al., “Seizures in children under five in a pediatric ward: Prevalence, associated factors and outcomes,” Open J. Pediatrics, vol. 11, no. 4, pp. 627-635, 2021.
81. N. A. Masento, M. Golightly, D. T. Field, L. T. Butler, and C. M. van Reekum, “Effects of hydration status on cognitive performance and mood,” Brit. J. Nutr., vol. 111, no. 10, pp. 1841-1852, May 2014.
82. J. M. Dickson et al., “The effects of dehydration on brain volume—preliminary results,” Int. J. Sports Med., vol. 26, no. 6, pp. 481-485, 2005.
83. M. Lauriola et al., “Neurocognitive disorders and dehydration in older patients: Clinical experience supports the hydromolecular hypothesis of dementia,” Nutrients, vol. 10, no. 5, May 2018, Art. no. 562.
84. W. F. Clark et al., “Urine volume and change in estimated GFR in a community-based cohort study,” Clin. J. Amer. Soc. Nephrol., vol. 6, no. 11, pp. 2634-2641, November 2011.
85 B. M. Popkin, K. E. D'Anci, and I. H. Rosenberg, “Water, hydration, and health,” Nutr. Rev., vol. 68, no. 8, pp. 439-458, August 2010.
86. K. E. D'Anci, F. Constant, and I. H. Rosenberg, “Hydration and cognitive function in children,” Nutr. Rev., vol. 64, no. 10, pp. 457-464, October 2006.
87. M. H. Gorelick, K. N. Shaw, and K. O. Murphy, “Validity and reliability of clinical signs in the diagnosis of dehydration in children,” Pediatrics, vol. 99, no. 5, p. e6, May 1997.
88. P. A. Phillips et al., “Reduced thirst after water deprivation in healthy elderly men,” New England J. Med., vol. 311, no. 12, pp. 753-759, September 1984.
89. N. S. Stachenfeld, C. A. Leone, E. S. Mitchell, E. Freese, and L. Harkness, “Water intake reverses dehydration associated impaired executive function in healthy young women,” Physiol. Behav., vol. 185, pp. 103-111, March 2018.
90. R. F. Reinoso, B. A. Telfer, and M. Rowland, “Tissue water content in rats measured by desiccation,” J. Pharmacological Toxicological Methods, vol. 38, no. 2, pp. 87-92, 1997.
91. H. P. Schwan and K. R. Foster, “Microwave dielectric properties of tissue. Some comments on the rotational mobility of tissue water,” Biophys. J., vol. 17, no. 2, pp. 193-197, 1977.
92. S. D. Meo et al., “The variability of dielectric permittivity of biological tissues with water content,” J. Electromagn. Waves Appl., vol. 36, no. 1, pp. 48-68, January 2022.
93. A. Shahzad, S. Khan, M. Jones, R. M. Dwyer, and M. O'Halloran, “Investigation of the effect of dehydration on tissue dielectric properties in EX vivo measurements,” Biomed Phys. Eng. Ep., vol. 3, no. 4, Jun. 26, 2017, Art. no. 45001.
94. D. A. Pollacco, L. Farina, P. S. Wismayer, L. Farrugia, and C. V. Sammut, “Characterization of the dielectric properties of biological tissues and their correlation to tissue hydration,” IEEE Trans. Dielectrics. Elect. Insul., vol. 25, no. 6, pp. 2191-2197, December 2018.
95. R. Balduino et al., “Feasibility of water content-based dielectric characterization of biological tissues using mixture models,” IEEE Trans. Dielectrics. Elect. Insul., vol. 26, no. 1, pp. 187-193, February 2019.
96. A. Shahzad, A. Elahi, P. Donlon, and M. O'Halloran, “A water dependent tissue dielectric model for estimation of in-vivo dielectric properties,” in Proc. IEEE MTT-S Int. Microw. Biomed Conf, 2020, pp. 1-3.
97. G. Maenhout, A. Santorelli, E. Porter, I. Ocket, T. Markovic, and B. Nauwelaers, “Effect of dehydration on dielectric measurements of biological tissue as function of time,” IEEE J. Electromagn. RF Microw. Med Biol., vol. 4, no. 3, pp. 200-207, September 2020.
98. A. Peyman, A. A. Rezazadeh, and C. Gabriel, “Changes in the dielectric properties of rat tissue as a function of age at microwave frequencies,” Phys. Med Biol., vol. 46, no. 6, pp. 1617-1629, June 2001.
99. E. Nyfors, “Industrial microwave sensors-A review,” Sens. Imag., vol. 1, no. 1, pp. 23-43, January 2000.
100. G. S. Raghavan and M. S. Venkatesh, “An overview of dielectric properties measuring techniques,” Can. Biosyst. Eng., vol. 47, no. 7, pp. 15-30, January 2005.
101. M. P. Robinson, J. Clegg, and D. A. Stone, “A novel method of studying total body water content using a resonant cavity: Experiments and numerical simulation,” Phys. Med Biol., vol. 48, no. 1, pp. 113-125, January 2003.
102. D. A. Stone and M. P. Robinsons, “Total body water content observations using cavity—perturbation techniques,” in Proc. High Freq. Postgraduate Student Colloq., 2003, pp. 31-34.
103. A. W. Kraszewski, S. O. Nelson, and T. S. You, “Use of a microwave cavity for sensing dielectric properties of arbitrarily shaped biological objects,” IEEE Trans. Microw. Theory Techn., vol. 38, no. 7, pp. 858-863, July 1990.
104. H. Choi et al., “Design and in vitro interference test of microwave noninvasive blood glucose monitoring sensor,” IEEE Trans. Microw. Theory Techn., vol. 63, no. 10, pp. 3016-3025, October 2015.
105. H. Choi, S. Luzio, J. Beutler, and A. Porch, “Microwave noninvasive blood glucose monitoring sensor: Human clinical trial results,” in Proc. IEEE MTT-S Int. Microw. Symp., Honololu, HI, USA, 2017, pp. 876-879.
106. M. Baghelani, Z. Abbasi, M. Daneshmand, and P. E. Light, “Noninvasive continuous-time glucose monitoring system using a chipless printable sensor based on split ring microwave resonators,” Sci. Rep., vol. 10, no. 1, 2020, Art. no. 12980.
107. M. Puentes, M. Maasch, M. Schussler, and R. Jakoby, “Frequency multiplexed 2-dimensional sensor array based on split-ring resonators for organic tissue analysis,” IEEE Trans. Microw. Theory Techn., vol. 60, no. 6, pp. 1720-1727, June 2012.
108. S. Hardinata, F. Deshours, G. Alquie, H. Kokabi, and F. Koskas, “Complementary split-ring resonators for non-invasive characterization of biological tissues,” in Proc. 18th Int. Symp. Antenna Technol. Appl. Electromagn., 2018, pp. 1-2.
109. D. C. Garrett and E. C. Fear, “Feasibility study of hydration monitoring using microwaves-Part 1: A model of microwave property changes with dehydration,” IEEE J. Electromagn. RF Microw. Med. Biol., vol. 3, no. 4, pp. 292-299, December 2019.
110. D. C. Garrett, J. R. Fletcher, D. B. Hogan, T. S. Fung, and E. C. Fear, “Feasibility study of hydration monitoring using microwaves-Part 2: Measurements of athletes,” IEEE J. Electromagn. RF Microw. Med. Biol., vol. 3, no. 4, pp. 300-307, December 2019.
111. B. C. Besler and E. C. Fear, “Microwave hydration monitoring: System assessment using fasting volunteers,” MDPI Sensors, vol. 21, no. 21, Oct. 20, 2021, Art. no. 6949.
112. R. Brendtke, M. Wiehl, F. Groeber, T. Schwarz, H. Walles, and J. Hansmann, “Feasibility study on a microwave-based sensor for measuring hydration level using human skin models,” PLoS One, vol. 11, no. 4, April 2016, Art. no. e0153145.
113. J. KilpijArvi, J. Tolvanen, J. Juuti, N. Halonen, and J. Hannu, “A non-invasive method for hydration status measurement with a microwave sensor using skin phantoms,” IEEE Sensors J., vol. 20, no. 2, pp. 1095-1104, January 2020.
114. A. F. McKinley, T. P. White, I. S. Maksymov, and K. R. Catchpole, “The analytical basis for the resonances and anti-resonances of loop antennas and meta-material ring resonators,” J. Appl. Phys., vol. 112, no. 9, November 2012, Art. no. 94911.
115. J. Wei, “Distributed capacitance of planar electrodes in optic and acoustic surface wave devices,” IEEE J. Quantum Electron., vol. 13, no. 4, pp. 152-158, April 1977.
116. F. Maradei and S. Caniggia, “Appendix A: Formulae for partial inductance calculation,” in Signal Integrity and Radiated Emission ofHigh-Speed Digital Systems. Chichester, U. K.: Wiley, 2008, pp. 481-486.
117. S. Bing, K. Chawang, and J.-C. Chiao, “A resonant coupler for subcutaneous implant,” MDPI Sensors, vol. 21, no. 23, December 2021, Art. no. 8141.
118. S. Bing, K. Chawang, and J. C. Chiao, “Resonant coupler designs for subcutaneous implants,” in Proc. IEEE Wireless Power Transfer Conf., 2021, pp. 1-4.
119. D. Andreuccetti, R. Fossi, and C. Petrucci, “An internet resource for the calculation of the dielectric properties of body tissues in the frequency range 10 Hz-100 GHz,” 1997. [Online]. Available: http://niremf.ifac. cnr.it/tissprop/
120. G. Maenhout, T. Markovic, I. Ocket, and B. Nauwelaers, “Effect of open-ended coaxial probe-to-tissue contact pressure on dielectric measurements,” MDPI Sensors, vol. 20, no. 7, April 2020, Art. no. 2060.
121. A. H. Sihvola and J. A. Kong, “Effective permittivity of dielectric mixtures,” IEEE Trans. Geosci. Remote Sens., vol. 26, no. 4, pp. 420-429, July 1988.
122. S. Evans, “Dielectric properties of ice and snow-a review,” J. Glaciology, vol. 5, no. 42, pp. 773-792, 1965.
123. K. Sasaki, E. Porter, E. A. Rashed, L. Farrugia, and G. Schmid, “Measurement and image-based estimation of dielectric properties of biological tissues-past, present, and future,” Phys. Med Biol., vol. 67, no. 14, July 2022, Art. no. 14TR01.
124. S. Gabriel, R. W. Lau, and C. Gabriel, “The dielectric properties of biological tissues: III parametric models for the dielectric spectrum of tissues,” Phys. Med Biol., vol. 41, no. 11, pp. 2271-2293, November 1996.
125. J. G. Lyng, L. Zhang, and N. P. Brunton, “A survey of the dielectric properties of meats and ingredients used in meat product manufacture,” Meat Sci., vol. 69, no. 4, pp. 589-602, 2005.
126. W. F. W. Southwood, “The thickness of the skin,” Plast. Reconstructive Surg., vol. 15, no. 5, pp. 423-429, May 1955.
127. Y. Lee and K. Hwang, “Skin thickness of korean adults,” Surg. Radiologic Anatomy, vol. 24, no. 3/4, pp. 183-189, 2002.
128. J. Sandby-Meller, T. Poulsen, and H. Wulf, “Epidermal thickness at different body sites: Relationship to age, gender, pigmentation, blood content, skin type and smoking habits,” Acta Dermato-Venereologica, vol. 83, no. 6, pp. 410-413, November 2003.
129. X. Huang et al., “Epidermal impedance sensing sheets for precision hydration assessment and spatial mapping,” IEEE Trans. Biomed Eng., vol. 60, no. 10, pp. 2848-2857, October 2013.
130. F. M. Hendriks, D. Brokken, C. W. J. Oomens, D. L. Bader, and F. P. T. Baaijens, “The relative contributions of different skin layers to the mechanical behavior of human skin in vivo using suction experiments,” Med Eng. Phys., vol. 28, no. 3, pp. 259-266, 2006.
131. Y. Feldman et al., “The electromagnetic response of human skin in the millimetre and submillimetre wave range,” Phys. Med Biol., vol. 54, no. 11, pp. 3341-3363, June 2009.

What is claimed is:

1. A system for noninvasive monitoring of water content comprising:
a radio-frequency (RF) planar resonant loop sensor comprising:
a planar loop antenna; and
an element disposed within and co-planar with a loop formed by the planar loop antenna; and
a detector configured to be connected with the RF planar resonant loop sensor to detect a near-field resonance.

2. The system of claim 1, wherein the water is disposed within organic material comprising human tissue, non-human animal tissue, or plant tissue.

3. The system of claim 1, wherein the water is disposed within an inorganic material.

4. The system of claim 1, wherein the system is configured to be disposed on or about a surface.

5. The system of claim 1, wherein the detector comprises a vector network analyzer, a scalar network analyzer, a spectrum analyzer, a phase-lock loop, or a frequency lock circuit.

6. The system of claim 5, wherein the system is configured to measure an |s₁₁| reflection coefficient.

7. The system of claim 5, wherein the system is configured to monitor water content over time by measuring a resonance twice or more in a selected time period.

8. The system of claim 5, wherein the system is configured to monitor water content over time by measuring a resonance continuously during a selected time period.

9. A kit comprising:
a radio-frequency (RF) planar resonant loop sensor comprising:
a planar loop antenna; and
an element disposed within and co-planar with a loop formed by the antenna; and
a detector configured to be connected with the RF planar resonant loop sensor to detect a near-field resonance; and
a device to secure the RF planar resonant loop sensor to a surface.

10. The kit of claim 9, wherein the water is disposed within an organic material comprising human tissue, non-human animal tissue, or plant tissue.

11. The kit of claim 9, wherein the water of is disposed within an inorganic material.

12. The kit of claim 9, wherein the system is configured to be disposed on or about a surface.

13. The kit of claim 9, wherein the detector comprises a vector network analyzer, a scalar network analyzer, a spectrum analyzer, a phase-lock loop, or a frequency lock circuit.

14. The kit of claim 13, wherein the system is configured to measure an |s₁₁| reflection coefficient.

15. The kit of claim 9, wherein the system is configured to monitor water content over time by measuring a resonance twice or more in a selected time period.

16. The kit of claim 9, wherein the system is configured to monitor water content over time by measuring a resonance continuously during a selected time period.

17. A method of measuring water content in a material comprising:
providing the material;
providing a system for noninvasive monitor or water content comprising:
a radio-frequency (RF) planar resonant loop sensor system comprising:
a planar loop antenna; and
an element disposed within and co-planar with a loop formed by the antenna; and
a detector configured to be connected with the RF planar resonant loop sensor to detect a near-field resonance;
disposing the loop on a surface of the material; and
measuring a near-field resonance with the system.

18. The method of claim 17, wherein the material comprises an organic material comprising human tissue, non-human animal tissue, or plant tissue.

19. The method of claim 17, wherein the material comprises an inorganic material.

20. The method of claim 17, wherein the step of measuring the near-field resonance with the system comprises measuring an |s₁₁| reflection coefficient.

21. The method of claim 17, further comprising measuring a near-field resonance twice or more in a selected time period.

22. The method of claim 17, further comprising measuring a near-field resonance continuously during a selected time period.
